(19) United States
(12) Patent Application Publication
(10) Pub. No.: US 2005/0282195 A1
(43) Pub. Date: Dec. 22, 2005

(54) Compositions, methods, and kits for (MIS)ligating oligonucleotides

(75) Inventors: Achim E. Karger, Foster City, CA (US); James E. Rozzelle, San Francisco, CA (US); Chien-Wei Chang, Sunnyvale, CA (US); Elena V. Bolchakova, Union City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/119,069

(22) Filed: Apr. 29, 2005

Related U.S. Application Data
(60) Provisional application No. 60/567,120, filed on Apr. 30, 2004.

(51) Int. Cl. C12Q 1/68; C12P 19/34; C12N 9/22

(52) U.S. Cl. 435/6; 435/91.2; 435/199

(57) ABSTRACT

Methods, reagents, and kits for (mis)ligating oligonucleotide probes or for identifying at least one target nucleotide are disclosed. One can enhance the generation of misligation product using a ligase under reaction conditions and with reagents where that particular ligase is prone to misligation. Alternatively, one can decrease or avoid generating misligation products using a particular ligase under reaction conditions and using reagents where that ligase is at least less prone to misligation. In certain embodiments, the recombinant ligase from Archaeoglobus fulgidus (Afu) is employed due to its unique misligation properties.

### CROSS-REFERENCE TO RELATED APPLICATIONS

This application claims a priority benefit under 35 U.S.C. § 119(e) from U.S. Patent Application No. 60/567,120, filed Apr. 30, 2004, which is incorporated herein by reference.

### FIELD

The present teachings generally relate to compositions, methods, and kits for ligating oligonucleotides. Compositions, methods and kits for increasing the generation of certain misligation products or for decreasing the generation of certain misligation products, are also disclosed.

### BACKGROUND

The DNA ligases are enzymes that typically catalyze phosphodiester bond formation between a 3′ OH group and a corresponding 5′ phosphate group of adjacent nucleotides at, for example, single-stranded breaks of duplex DNA. Some ligases reportedly catalyze phosphodiester bond formation between DNA and RNA molecules or can ligate together single stranded sequences, i.e., blunt end ligation rather than template-dependent ligation. DNA ligases may be divided into two categories, the NAD⁺-dependent ligases and the ATP-dependent ligases, although all ligases are believed to employ a common reaction mechanism. Generally, the known eubacterial ligases use NAD⁺ as a cofactor, while the known ligases from eukaryotes and archeae, including the known ligases from viruses that infect eukaryotes or archeae, and even bacteriophages typically use ATP as a cofactor, although some variations from this generalized scheme have been reported.

Ligase catalyzed reactions form the basis for several current assay techniques, for example but not limited to, the oligonucleotide ligation assay (OLA), the ligase chain reaction (LCR), the ligase detection reaction (LDR) and combination assays such as the OLA coupled with the polymerase chain reaction (PCR), e.g., OLA-PCR and PCR-OLA, the Combined Chain Reaction (CCR; a combination of PCR and LCR) and PCR-LDR (see, e.g., Landegren et al., Science 241:1077-80, 1988; Barany, Proc. Natl. Acad. Sci. 88:189-93, 1991; Grossman et al., Nucl. Acids Res. 22(21):4527-34, 1994; Bi and Stambrook, Nucl. Acids Res. 25(14):2949-51, 1997; Zirvi et al., Nucl. Acids Res., 27(24):e40, 1999; U.S. Pat. No. 4,988,617; and PCT Publication Nos. WO 97/31256 and WO 01/92579. Such assays have been used for single nucleotide polymorphism (SNP) analysis, SNP genotyping, mutation detection, identification of single copy genes, detecting microsatellite repeat sequences, and DNA adduct mapping, among other things.

The accuracy of these ligation-based assays generally depend on (1) the fidelity of the ligase to distinguish (a) potential ligation sites where both the upstream and downstream probes are correctly base-paired with the template to which they are hybridized from (b) potential ligation sites where at least one nucleotide of at least one probe is not correctly base-paired with the template, sometimes referred to as mismatched, (2) reaction conditions that preclude or minimize hybridization of mismatched probes, or (iii) both (see, e.g., Landegren et al., Science 241:1077-80, 1988; Barany, Proc. Natl. Acad. Sci. 88:189-93, 1991). Generally, a high fidelity ligase, i.e., one that catalyzes the ligation of correctly base-paired sequences but does not ligate mismatched sequences is desired (see, e.g., Barany, Proc. Natl. Acad. Sci. 88:189-93, 1991; Luo et al., Nucl. Acids Res. 24(14):3071-78, 1996; and Housby et al., Nucl. Acids Res. 28(3):e10, 2000). Additionally, since these ligation-based assays typically include thermocycling, thermostable ligases are generally preferred (see, e.g., Cao, Trends in Biotech 22(1): 38-44, 2004).

While these ligation-based assays rely in part on the fidelity of the enzyme to distinguish properly base-paired from mismatched probes, ligase fidelity is reportedly highly variable, depending on the properties of the particular enzyme, the identity of the mismatched nucleotides, the location of the mismatched nucleotides relative to the ligation junction (also known as the ligation site), the sequence context around the ligation junction, cofactors, and reaction conditions, among other things. The fidelity of several known ligases, based on for example the evaluation of mismatch ligation or ligation rates, has been reported. For example, the NAD⁺-dependent ligase from the hyperthermophilic bacteria Aquifex aeolicus reportedly generates detectable 3′ misligation products with C:A, T:G, and G:T mismatches (Tong et al., Nucl. Acids Res. 28(6):1447-54, 2000); a partially purified preparation of bovine DNA ligase III reportedly generated detectable 3′ misligation products with C:T, G:T, and T:G mismatches, while human ligase I generated detectable 3′misligation products with C:T and G:T mismatches, but not T:G mismatches (Husain et al., J. Biol. Chem. 270(16):9683-90, 1995); and the DNA ligase from the thermophilic bacteria Thermus thermophilus (Tth) reportedly generates detectable levels of 3′ misligation products with T:G and G:T mismatches (Luo et al., Nucl. Acids Res. 24(14):3071-78, 1996). Bacteriophage T4 DNA ligase reportedly generates detectable misligation products with a wide range of mismatched substrates and appears to have lower fidelity than Thermus species ligases by at least one to two orders of magnitude (Landegren et al., Science 241:1077-80, 1988; Tong et al., Nucl. Acids Res. 27(3):788-94, 1999).

The reliability of certain ligation-based assays, particularly those that employ two or more alternate allele- or target-specific oligonucleotides, may be affected by the tendency of the ligase to generate background misligation products. For example without limitation, an illustrative OLA for analyzing a biallelic SNP site may employ a single species of downstream oligonucleotide (sometimes referred to as a locus-specific oligo or LSO) and two alternate species of upstream oligonucleotides (sometimes referred to as allele-specific oligos or ASOs) that differ in their template-specific portions by, for example, the 3′ terminal nucleotide, with each of the upstream oligo species corresponding to one of the two alternate SNP site alleles being interrogated. Depending on, among other things, the ligase employed; the identity of the mismatched nucleotide(s) and the “corresponding” template nucleotide(s); the sequence context around the ligation junction; the concentration of template, ligation probes, and/or ligase; and the ligation reaction conditions, a misligation product may be formed (or the generation of misligation products may also be avoided or at least minimized). Depending, at least in part, on the amount of misligation product formed, the sample being interrogated, and the sensitivity of the detection technique employed, the misligation product may result in an erroneously characterization of the sample.

Ligase fidelity studies to date generally demonstrate a high degree of substrate specificity with certain mismatches, while misligation products are generated with other mismatched substrate-ligation probe complexes. Thus, ligases tend to have characteristic misligation patterns that can, at least in certain instances, be distinguishing (see, e.g., Sriskanda and Shuman, Nucl. Acids Res. 26(15):3536-41; and Tong et al., Nucl. Acids Res. 28(6):1447-54, 2000). Thus, in certain instances it may be desirable to have a ligase that either will or will not generate detectable misligation products, based on the intended application.

### SUMMARY

The present teachings are directed to methods, reagents, and kits for ligating oligonucleotide probes, that can but need not comprise nucleotide analogs. In certain embodiments, the generation of misligation products is desired, while in other embodiments, the generation of misligation products is at least decreased. One can enhance the generation of misligation product using a ligase under reaction conditions and with reagents where that particular ligase is prone to misligation. Alternatively, one can decrease or avoid generating misligation products using a particular ligase under reaction conditions and using reagents where that ligase is at least less prone to misligation. While characterizing the ligase derived from the hyperthermostable archaea Archaeoglobus fulgidus (Afu), the applicants made the unexpected finding that the misligation pattern of isolated recombinant Afu ligase is markedly different from other known ligases. For example, using M13-derived oligonucleotide probes and synthetic target nucleic acid sequences, the ligases from Thermus aquaticus (Taq) and Thermus species AK16D (“AK16D”) were much more prone to 3′ G:T misligation compared to Afu ligase, while Afu ligase was more prone to misligations comprising a C nucleotide than either of these Thermus ligases under the same conditions (see, e.g., Table 1). Applicants are unaware of any published reports of a thermostable ligase that can effectively discriminates against 3′T:G mismatched substrates, but can produce detectable misligation products with 3′T:C substrates (see also co-filed U.S. Provisional Patent Application Ser. No. 60/567,068, filed Apr. 30, 2005 for “Methods and Kits for Identifying Target Nucleotides in Mixed Populations,” by Karger and Bolchakova, and co-filed U.S. Provisional Patent Application Ser. No. 60/567,396, filed Apr. 30, 2004 for “Methods and Kits for Methylation Detection,” by Andersen et al).

In certain embodiments of the current teachings, an isolated thermostable recombinant polypeptide, derived from the hyperthermophilic archaea Archaeoglobus fulgidus, possessing ligase activity is disclosed. In certain embodiments, methods for producing that recombinant thermostable ligase are disclosed.

In certain embodiments, compositions comprising isolated polypeptide are disclosed that efficiently misligate adjacently hybridized oligonucleotides when the 3′ terminus of the upstream probe is a T and the corresponding template nucleotide is a C (3′T:C), but does not efficiently misligate adjacently hybridized suitable oligonucleotides when the nucleotide on the 3′ terminus of the upstream probe is a T and the corresponding template nucleotide is a G (3′T:G). In certain embodiments, the efficiently misligates refers to a ligation rate ratio, relative to Taq ligase or AK16D ligase, of at least 5:1, at least 10:1, or at least 20:1 when the same oligos and template are used under the same ligation reaction conditions.

In certain embodiments, the does not efficiently misligate refers to a ligation rate ratio of at least 1:10, at least 1:20, or at least 1:30, relative to Taq ligase or AK16D ligase when the same oligos and template are used under the same ligation reaction conditions. In certain embodiments, the efficiently misligates refers to a ligation rate ratio of at least 5:1, at least 10:1 or at least 20:1, relative to Taq ligase or AK16D ligase when the same oligos and template are used under the same ligation reaction conditions; and the does not efficiently misligate refers to a ligation rate ratio of at least 1:10, at least 1:20, or at least 1:30, relative to Taq ligase or AK16D ligase when the same oligos and template are used under the same ligation reaction conditions.

In certain embodiments, methods for generating ligation products are disclosed, wherein a ligation reaction composition comprising at least one probe set, at least one target nucleic acid sequence, and Afu ligase, including but not limited to at least one enzymatically active mutant or variant thereof, is formed and incubated under appropriate conditions to generate at least one ligation product. Methods for amplifying such ligation products are disclosed. Methods for generating at least one digested ligation product, at least one amplified digested ligation product, or combinations thereof, are also disclosed. In certain embodiments, amplifying is performed before the ligating, before the digesting, or before the ligating and before the digesting. In certain embodiments, the amplifying is performed after the ligation, after the digesting, or after the ligating and after the digesting. In certain embodiments, at least one ligation product, at least one ligation product surrogate (e.g., at least one amplified ligation product, at least one digested ligation product, at least one amplified digested ligation product, where an “amplified digested ligation product” includes both a ligation product that is first amplified, then digested; and a ligation product that is first digested, then amplified), or combinations thereof are detected and evaluated to identify the presence of and/or the quantity of a specific target nucleotide in the sample.

In certain embodiments, methods for generating misligation products are disclosed, wherein a ligation reaction composition comprising at least one probe set, at least one target nucleic acid sequence, and Afu ligase, including but not limited to at least one enzymatically active mutant or variant thereof, is formed and incubated under appropriate conditions to generate at least one misligation product. Methods for amplifying such misligation products are disclosed. Methods for generating at least one digested misligation product, at least one amplified digested misligation product, or combinations thereof, are also disclosed. In certain embodiments, amplifying is performed before the ligating, before the digesting, or before the ligating and before the digesting. In certain embodiments, the amplifying is performed after the ligation, after the digesting, or after the ligating and after the digesting. In certain embodiments, at least one misligation product, at least one misligation product surrogate (e.g., at least one amplified misligation product, at least one digested misligation product, at least one amplified digested misligation product, where an “amplified digested misligation product” includes both a misligation product that is first amplified, then digested; and a misligation product that is first digested, then amplified), or combinations thereof are detected and analyzed to identify the presence and/or the quantity of a specific target nucleotide in the sample.

In certain embodiments, genomic DNA (gDNA) serves as the ligation template. In certain embodiments, gDNA is amplified and the amplified DNA serves as the ligation template, either in addition to or in place of the gDNA. Within the scope of the teachings are large-scale multiplex analyses of target nucleic acid sequences, including multiplex ligation steps, multiplex amplification steps, or both multiplex ligation steps and multiplex amplification steps.

In certain embodiments, methods for generating at least one ligation product are provided, wherein at least one target nucleic acid sequence is combined with at least one probe set for each target sequence to be interrogated, and at least one ligase to form a ligation reaction composition. The at least one probe set comprises at least one first probe comprising at least one first target-specific portion, and at least one second probe comprising at least one second target-specific portion. The probes in each set are suitable for ligation together when hybridized adjacent to one another on a complementary target sequence, e.g., the end of the upstream probe nearest the ligation site includes a 3′ OH group and the end of the downstream probe nearest the ligation site includes a 5′ phosphate group. This ligation reaction composition is subjected to at least one cycle of ligation, wherein adjacently hybridizing probes, under appropriate conditions, are ligated to one another to generate at least one ligation product. The ligation product thus comprises the target-specific portions. In certain embodiments, at least one probe in a probe set further comprises at least one hybridization tag, at least one primer-binding portion, at least one reporter group, or combinations thereof; and the ligation of such a probe set comprises the target-specific portions and at least one hybridization tag, at least one primer-binding portion, at least one reporter group, or combinations thereof, as appropriate. Those in the art will understand that, depending on the ligase and the reaction conditions, the at least one ligation product can but need not include at least one misligation product.

In certain embodiments, at least one ligation product comprising at least one primer-binding portion is combined with at least one primer and a polymerase to form an amplification reaction composition. In certain embodiments, the amplification reaction composition comprises at least one primer set. The amplification reaction composition is subjected to at least one cycle of amplification to generate at least one amplified ligation product. In certain embodiments, the target nucleic acid sequences are first amplified and then combined with at least one probe set to form a ligation reaction composition. Such ligation reaction compositions are subjected to at least one cycle of ligation, wherein adjacently hybridized probes, under appropriate conditions, are ligated together to form a ligation product. Those in the art will understand that, depending on the ligase and the reaction conditions, the at least one amplified ligation product can but need not include at least one amplified misligation product.

In certain embodiments, at least one first probe, at least one second probe, or at least one first probe and at least one second probe of a probe set further comprise at least one hybridization tag designed to allow hybridization with corresponding hybridization tag complements, such as capture oligonucleotides attached to a support; or to provide a unique molecular weight or length, or mobility, including without limitation, electrophoretic mobility, particularly when the hybridization tag comprises at least one mobility modifier. In certain embodiments, at least one probe further comprises, at least one reporter group, including without limitation at least one fluorescent reporter group and/or at least one affinity tag; at least one mobility modifier; at least part of at least one reporter probe binding portion; or combinations thereof.

In certain embodiments, at least one primer comprises at least one hybridization tag designed to allow hybridization with corresponding hybridization tag complements, such as capture oligonucleotides attached to a support; or to provide a unique molecular weight or length, or mobility, including without limitation, electrophoretic mobility, particularly when the hybridization tag comprises at least one mobility modifier. In certain embodiments, at least one primer further comprises at least one reporter group, including without limitation at least one fluorescent reporter group and/or at least one affinity tag; at least one mobility modifier; at least part of at least one reporter probe binding portion; or combinations thereof.

In certain embodiments, single-stranded amplification products comprising hybridization tags, suitable for hybridization with a support comprising corresponding hybridization tag complements, can be generated by several alternate methods including, without limitation, asymmetric PCR, asymmetric reamplification, nuclease digestion, and chemical denaturation. Detailed descriptions of such processes can be found, among other places, in Ausbel et al., Current Protocols in Molecular Biology (1993) including supplements through April 2004, John Wiley & Sons (hereinafter “Ausbel et al.”), Novagen Strandase™ Kit insert, Sambrook et al., Molecular Cloning, A Laboratory Manual, Cold Spring Harbor Press (1989; hereinafter “Sambrook et al.”), Little et al., J. Biol. Chem. 242:672 (1967) and PCT Publication No. WO 01/92579.

In certain embodiments, methods are disclosed for generating at least one ligation product, comprising: at least one step for interrogating at least one target nucleotide; and at least one step for generating at least one ligation product. Certain embodiments of these methods further comprise at least one step for amplifying the at least one ligation product; at least one step for digesting the at least one amplified ligation product; at least one step for digesting the at least one ligation product; at least one step for amplifying the at least one digested ligation product; or combinations thereof.

Those skilled in the art will appreciate that the at least one step for interrogating can be performed using the probes and probe sets disclosed herein; that the at least one step for generating at least one (mis)ligation product can be performed using the ligation means and/or ligation techniques disclosed herein; that the at least one step for generating at least one amplified (mis)ligation product can be performed using the amplification means, amplification techniques, ligation means, and/or ligation techniques disclosed herein, including combinations thereof; that the at least one step for generating at least one digested (mis)ligation product or at least one (mis)ligation product surrogate can be performed using the digesting means and/or digestion techniques disclosed herein; and that the at least one step for identifying at least one target nucleotide can be performed using the identifying and determining means and techniques disclosed herein. In certain embodiments, identifying can, but need not, comprise substeps for separating, detecting, evaluating, analyzing, comparing, or combinations thereof. In certain embodiments, the separating is performed independently, i.e., is not a substep of the identifying. Certain of the disclosed methods and kits comprise at least two separating steps and/or at least two separating means, and can, but need not, include at least two separating techniques.

In certain embodiments, methods are disclosed for generating at least one misligation product, comprising: at least one step for interrogating at least one target nucleotide; and at least one step for generating at least one misligation product. Certain embodiments of these methods further comprise at least one step for amplifying the at least one misligation product; at least one step for digesting the at least one amplified misligation product; at least one step for digesting the at least one misligation product; at least one step for amplifying the at least one digested misligation product; or combinations thereof.

Method for identifying at least two target nucleotides in a sample are also provided. In certain embodiments, at least one first target nucleotide and at least one second target nucleotide are identified as follows. A first ligation reaction composition comprising (a) at least one first target nucleic acid sequence, (b) at least one first ligation probe set comprising at least one first probe and at least one second probe, wherein the at least one first probe comprises at least one target-specific portion and the at least one second probe comprises at least one target-specific portion, and (c) at least one first ligase is formed. A second ligation reaction composition comprising (a) at least one second target nucleic acid sequence, (b) at least one second ligation probe set comprising at least one first probe and at least one second probe, wherein the at least one first probe comprises at least one target-specific portion and the at least one second probe comprises at least one target-specific portion, and (c) at least one second ligase is formed. The first and the second ligation reaction compositions can be prepared in parallel, i.e., at the same or essentially the same time, or they may be prepared separately.

The first ligation reaction composition and the second ligation reaction compositions are each subjected to at least one cycle of ligation, either in parallel or separately, and at least one first ligation product is generated. In certain embodiments, the first and/or second reaction compositions are subjected to a multiplicity of cycles of ligation. The at least one first target nucleotide, the at least one second target nucleotide, or the at least one first target nucleotide and the at least one second target nucleotide in the sample are identified, either separately or in parallel, typically by detecting and evaluating the corresponding ligation products or their surrogates.

In certain embodiments, at least one of the ligases is Afu ligase (including enzymatically active mutant or variants of Afu ligase). In certain embodiments, at least one of the ligases is a thermostable ligase, including without limitation, a ligase derived or obtained from a member of the species Thermus (including enzymatically active mutant or variants the thermostable ligase). In certain embodiments, at least one ligase is Afu ligase and at least one other ligase is a thermostable ligase, but not Afu ligase (including enzymatically active mutant or variants of Afu ligase and at least one other ligase). In certain embodiments, at least one target nucleotide is amplified prior to or contemporaneous with the ligation reaction. In certain embodiments, at least one ligation product is amplified to generate an amplified ligation product. In certain embodiments, at least one amplified ligation product or other ligation product surrogate is digested by, for example, a nuclease.

In certain embodiments, at least one first ligation product, at least one second ligation product, or the at least one first ligation product and the at least one second ligation product, includes at least one primer-binding portion, at least one reporter group, at least one mobility modifier, at least one hybridization tag, at least one reporter probe binding portion, at least one affinity tag, or combinations thereof. In certain embodiments, at least one amplified ligation product includes at least one primer-binding portion, at least one reporter group, at least one mobility modifier, at least one hybridization tag, at least one reporter probe-binding portion, at least one affinity tag, or combinations thereof. In certain embodiments, identifying comprises detecting at least one reporter group of at least one first ligation product (and/or its surrogate) and at least one reporter group of at least one second ligation product (and/or its surrogate), and quantifying the amount of the first ligation product that is generated, the amount of the second ligation that is generated. In certain embodiments, the amounts of generated ligation products are compared and/or ratios are calculated. In certain embodiments, identifying includes detecting at least one reporter probe, at least part of a reporter probe, or at least one reporter probe and at least part of a reporter probe.

The instant teachings also provide kits designed to expedite performing the subject methods. Kits serve to expedite the performance of the disclosed methods by assembling two or more components required for carrying out the methods. In certain embodiments, kits contain components in pre-measured unit amounts to minimize the need for measurements by end-users. In certain embodiments, kits include instructions for performing one or more of the disclosed methods. In certain embodiments, kit components are optimized to operate in conjunction with one another.

The disclosed kits may be used to generate at least one ligation product, at least one amplified ligation product, at least one amplified digested ligation product, at least one digested ligation product, or combinations thereof. The disclosed kits may also be used to generate at least one misligation product, at least one amplified misligation product, at least one amplified digested misligation product, at least one digested misligation product, or combinations thereof. In certain embodiments, the instant kits comprise Afu ligase, at least one second ligase, at least one polymerase, at least one nuclease, at least one reporter group, at least one mobility modifier, at least one affinity tag, at least one hybridization tag, at least one probe set, at least one primer, or combinations thereof. In certain embodiments, the Afu ligase comprises at least one enzymatically active mutant of Afu ligase, at least one enzymatically active variant of Afu ligase, or combinations thereof, including but not limited to enzymatically active mutants or variants of recombinant Afu ligase. In certain embodiments, kits are disclosed that comprise at least one means for ligating, at least one means for amplifying, at least one means for separating, at least one means for digesting, at least one detection means, at least one identifying means, or combinations thereof.

### BRIEF DESCRIPTION OF THE DRAWINGS

FIG. 1: depicts two panels of illustrative electropherograms, each showing ligation product peaks, including misligation product peaks, generated according to one of the disclosed methods using human gDNA target nucleic acid sequences (Sample # 35, 41, 45 and 158) and Y-chromosome SNP sites (e.g., Y1, Y6, Y10, Y16, Y20, Y40, Y46, Y 68, and Y84), as described in Example 7. The top panel of four electropherograms shows the (mis)ligation product peaks obtained with each of the four samples using Afu ligase and the bottom panel of four electropherograms shows the (mis)ligation product peaks obtained with each of the four samples using Taq ligase. Peaks marked with an arrow and “% MM” indicate misligation product peaks and the percent of misligation product generated compared to the match ligation product. The x-axis (numbered across the top) represents size in nucleotide length relative to a LIZ 120 size standard and the y-axis represents fluorescent intensity in relative fluorescence units (RFU).

FIG. 2: is a graph showing the effect of varying concentrations of the divalent cation cofactors, magnesium (Mg; shown as diamonds) and manganese (Mn; shown as triangles) on the ligation rate of Afu ligase, as described in Example 8. The x-axis represents the cofactor concentration in mM and the y-axis represents the ligation rate in fmol/min.

FIG. 3: is a semi-log graph depicting thermal decay curves for Afu ligase (shown as diamonds) and Taq ligase (shown as squares), as described in Example 9. The x-axis represents the incubation time at 95° C. in minutes. The y-axis represents the percent ligase activity remaining in log scale.

### DESCRIPTION OF EXEMPLARY EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. In the event that one or more of the incorporated literature and similar materials differs from or contradicts this application, including but not limited to defined terms, term usage, described techniques, or the like, this application controls.

### I. DEFINITIONS

The term “adjacently hybridized oligonucleotides” refers to the final location of two nucleic acid sequences (typically oligonucleotide probes), relative to a target nucleic acid sequence or template prior to ligation, regardless of how they arrived at that final location. Under appropriate conditions, adjacently hybridized oligonucleotides can be ligated together to form a (mis)ligation product. In certain embodiments, two nucleic acid sequences hybridize in a juxtaposed fashion such that the 3′-end of the upstream oligonucleotide (relative to the template in a 3′-5′ orientation, left to right) is on one side of a ligation junction, also referred to as a ligation site, and the 5′-end of the downstream probe is on the opposing end of the ligation junction. In certain embodiments, two probes of a probe set hybridize to the corresponding template but are not immediately adjacent. Before the two probes can be ligated together, an intermediate gap-filling step such a primer extension is be performed. In certain embodiments, a series of three or more oligonucleotides are ligated together on the template. In this context, the term oligonucleotide, such as an oligonucleotide probe can include a nucleic acid sequence with at least two linked nucleotides or at least one nucleotide linked to at least one adjacent nucleotide analog.

The term “or combinations thereof′” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to at least one specific relationship between the elements to which the term refers. For example, at least one first probe of a particular probe set corresponds to at least one second probe of the same probe set, and vice versa. At least one primer is designed to anneal with the primer-binding portion of at least one corresponding probe, at least one corresponding (mis)ligation product, at least one corresponding amplified (mis)ligation product, at least one corresponding digested (mis)ligation product, at least one corresponding digested amplified (mis)ligation product, or combinations thereof. The target-specific portions of the probes of a particular probe set are designed to hybridize with a complementary or substantially complementary region of the corresponding target nucleic acid sequence. A particular affinity tag binds to the corresponding affinity tag, for example but not limited to, biotin binding to streptavidin. A particular hybridization tag anneals with its corresponding hybridization tag complement; and so forth.

The term “that efficiently misligates” or equivalents are relative terms that refer to the amount of ligation product, in this case misligation product, that a given ligase generates under specified reaction conditions, including but not limited to, the enzyme concentration, the substrate and substrate concentration, the template and template concentration, the cofactors and their concentrations, including without limitation, ATP and/or NAD⁺, metal cofactors, buffer, reaction temperature, and the like, compared to the amount of misligation product that a second ligase generates under similar conditions. In certain embodiments, a ligase is said to efficiently misligate when its misligation rate ratio relative to Thermus aquaticus (Taq) ligase or by Thermus species AK16D (AK16D) ligase under the reaction conditions and using the analytical techniques described in Example 4 is: at least 5:1; at least 7:1; at least 10:1; at least 15:1; at least 20:1; at least 25:1; or at least 30:1 (i.e., ligase “X”/Taq ligase or ligase “X”/AK16D).

Those in the art appreciate that the term “does not efficiently misligate” and similar terminology is not the equivalent of the term “that efficiently misligates” or similar terms as those terms are used herein. Rather, the term “does not efficiently misligate” or equivalents are essentially the opposite of terms such as “efficiently misligates”. Thus, in certain embodiments, a ligase does not efficiently misligate when its misligation rate ratio relative to that of Thermus aquaticus (Taq) ligase or Thermus species AK16D (AK16D) ligase under the reaction conditions and using the analytical techniques described in Example ______ is: at least 1:5; at least 1:7; at least 1:10; at least 1:15; at least 1:20; at least 1:25; or at least 1:30 (i.e., ligase “X”/Taq ligase or ligase “X”/AK16D). If, for example, the misligation product of exemplary ligase “X” is not detectable but the misligation product of Taq ligase is detectable under the reaction conditions and using the analytical techniques described in Example 4, then by definition ligase “X” does not efficiently misligate relative to Taq ligase.

The term “enzymatically active mutants or variants thereof′ and equivalent terms when used in reference to one or more enzyme, such as one or more polymerase, one or more ligase, one or more nuclease, or the like, refers to one or more polypeptide derived from the corresponding enzyme that retains at least some of the desired enzymatic activity, such as ligating, amplifying, or digesting, as appropriate. Also within the scope of this term are: enzymatically active fragments, including but not limited to, cleavage products, for example but not limited to Klenow fragment, Stoffel fragment, or recombinantly expressed fragments and/or polypeptides that are smaller in size than the corresponding enzyme; mutant forms of the corresponding enzyme, including but not limited to, naturally-occurring mutants, such as those that vary from the “wild-type” or consensus amino acid sequence, mutants that are generated using physical and/or chemical mutagens, and genetically engineered mutants, for example but not limited to random and site-directed mutagenesis techniques; amino acid insertions and deletions, and changes due to nucleic acid nonsense mutations, missense mutations, and frameshift mutations (see, e.g., Sriskanda and Shuman, Nucl. Acids Res. 26(2):525-31, 1998; Odell et al., Nucl. Acids Res. 31(17):5090-5100, 2003); reversibly modified nucleases, ligases, and polymerases, for example but not limited to those described in U.S. Pat. No. 5,773,258; biologically active polypeptides obtained from gene shuffling techniques (see, e.g., U.S. Pat. Nos. 6,319,714 and 6,159,688), splice variants, both naturally occurring and genetically engineered, provided that they are derived, at least in part, from one or more corresponding enzymes; polypeptides corresponding at least in part to one or more such enzymes that comprise modifications to one or more amino acids of the native sequence, including without limitation, adding, removing or altering glycosylation, disulfide bonds, hydroxyl side chains, and phosphate side chains, or crosslinking, provided such modified polypeptides retain at least some appropriate catalytic activity; and the like.

The skilled artisan will readily be able to measure enzymatic activity using an appropriate well-known assay. Thus, an appropriate assay for polymerase catalytic activity might include, for example, measuring the ability of a variant to incorporate, under appropriate conditions, rNTPs or dNTPs into a nascent polynucleotide strand in a template-dependent manner. Likewise, an appropriate assay for ligase catalytic activity might include, for example, the ability to ligate adjacently hybridized oligonucleotides comprising appropriate reactive groups, such as disclosed herein. Protocols for such assays may be found, among other places, in Sambrook et al., Sambrook and Russell, Molecular Cloning, Third Edition, Cold Spring Harbor Press (2000) (hereinafter “Sambrook and Russell”), Ausbel et al., and Housby and Southern, Nucl. Acids Res. 26:4259-66, 1998).

The terms “fluorophore” and “fluorescent reporter group” are intended to include any compound, label, or moiety that absorbs energy, typically from an illumination source or energy transfer, to reach an electronically excited state, and then emits energy, typically at a characteristic wavelength, to achieve a lower energy state. For example but without limitation, when certain fluorophores are illuminated by an energy source with an appropriate excitation wavelength, typically an incandescent or laser light source, photons in the fluorophore are emitted at a characteristic fluorescent emission wavelength. Fluorophores, sometimes referred to as fluorescent dyes, may typically be divided into families, such as fluorescein and its derivatives; rhodamine and its derivatives; cyanine and its derivatives; coumarin and its derivatives; Cascade Blue™ and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa Fluor 488, Alexa Fluor 532, Alexa Fluor 546, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine™), carboxy tetramethylrhodamine (TAMRA™), carboxy-X-rhodamine (ROX™), LIZ™, VIC™, NED™, PET™, SYBR, PicoGreen, RiboGreen, and the like. Descriptions of fluorophores and their use, can be found in, among other places, R. Haugland, Handbook of Fluorescent Probes and Research Products, 9^{th} ed. (2002), Molecular Probes, Eugene, Oreg. (hereinafter “Molecular Probes Handbook”); M. Schena, Microarray Analysis (2003), John Wiley & Sons, Hoboken, N.J.; Synthetic Medicinal Chemistry 2003/2004 Catalog, Berry and Associates, Ann Arbor, Mich.; U.S. Pat. No. 6,025,505; G. T. Hermanson, Bioconjugate Techniques, Academic Press, San Diego, Calif. (1996)(hereinafter “Bioconjugate Techniques”); and Glen Research 2002 Catalog, Sterling, Va. Near-infrared dyes are expressly within the scope of the terms fluorophore and fluorescent reporter group, as are combination labels, such as combinatorial fluorescence energy transfer tags (see, e.g. Tong et al., Nat. Biotech. 19:756-59, 2001).

The term “hybridization tag” as used herein refers to an oligonucleotide sequence that can be used for separating the element (e.g., ligation products, surrogates, ZipChutes, etc.) of which it is a component or to which it is bound, including without limitation, bulk separation; for tethering or attaching the element to which it is bound to a substrate, which may or may not include separating; for annealing a hybridization tag complement that may comprise at least one moiety, such as a mobility modifier, one or more reporter groups, and the like; or combinations thereof. In certain embodiments, the same hybridization tag is used with a multiplicity of different elements to effect: bulk separation, substrate attachment, or combinations thereof. A “hybridization tag complement” typically refers to at least one oligonucleotide that comprises at least one sequence of nucleotides that are at least substantially complementary to and hybridize with the corresponding hybridization tag. In various embodiments, hybridization tag complements serve as capture moieties for attaching at least one hybridization tag:element complex to at least one substrate; serve as “pull-out” sequences for bulk separation procedures; or both as capture moieties and as pull-out sequences. In certain embodiments, at least one hybridization tag complement comprises at least one reporter group and serves as a label for at least one ligation product, at least one ligation product surrogate, or combinations thereof. In certain embodiments, determining comprises detecting one or more reporter groups on or attached to at least one hybridization tag complement or at least part of a hybridization tag complement.

Typically, hybridization tags and their corresponding hybridization tag complements are selected to minimize: internal self-hybridization; cross-hybridization with different hybridization tag species, nucleotide sequences in a reaction composition, including but not limited to gDNA, different species of hybridization tag complements, target-specific portions of probes, and the like; but should be amenable to facile hybridization between the hybridization tag and its corresponding hybridization tag complement. Hybridization tag sequences and hybridization tag complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65 (1998)). Descriptions of hybridization tags can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein); and Gerry et al., J. Mol. Biol. 292:251-262 (1999; referred to as “zip-codes” and “zip-code complements” therein). Those in the art will appreciate that a hybridization tag and its corresponding hybridization tag complement are, by definition, complementary to each other and that the terms hybridization tag and hybridization tag complement are relative and can essentially be used interchangeably in most contexts.

Hybridization tags can be located on at least one end of at least one probe, at least one primer, at least one ligation product, at least one ligation product surrogate, or combinations thereof; or they can be located internally. In certain embodiments, at least one hybridization tag is attached to at least one probe, at least one primer, at least one ligation product, at least one ligation product surrogate, or combinations thereof, via at least one linker arm. In certain embodiments, at least one linker arm is cleavable.

In certain embodiments, hybridization tags are at least 12 bases in length, at least 15 bases in length, 12-60 bases in length, or 15-30 bases in length. In certain embodiments, at least one hybridization tag is 12, 15, 20, 21, 22, 23, 24, 25, 26, 27, 28, 29, 30, 45, or 60 bases in length. In certain embodiments, at least two hybridization tag:hybridization tag complement duplexes have melting temperatures that fall within a Δ Tₘ range (Tₘₐₓ-Tₘᵢₙ) of no more than 10° C. of each other. In certain embodiments, at least two hybridization tag:hybridization tag complement duplexes have melting temperatures that fall within a Δ Tₘ range of 5° C. or less of each other.

In certain embodiments, at least one hybridization tag complement comprises at least one reporter group, at least one mobility modifier, at least one reporter probe binding portion, or combinations thereof. In certain embodiments, at least one hybridization tag complement is annealed to at least one corresponding hybridization tag and, subsequently, at least part of that hybridization tag complement is released and detected.

The term “ligation product” refers to a molecule that is generated when an internucleotide linkage is formed between two corresponding probes by the action of a ligase. Those in the art understand that, under certain conditions, such an internucleotide linkage can be formed between a pair of matched probes, a pair of mismatched probes (that is at least one of the two probes comprises at least one nucleotide or nucleotide analog that is mismatched with the corresponding template), or both. Thus, the term (mis)ligation is used herein to collectively refer to at least one match ligation, at least one mismatch ligation (sometimes referred to as misligation), or at least one match ligation and at least one misligation. Hence, by way of illustration, at least one “(mis)ligation product” refers to at least one ligation product, at least one misligation product, or at least one ligation product and at least one misligation product; at least one “(mis)ligation product surrogate” refers to at least one ligation product surrogate, at least one misligation product surrogate, or at least one ligation product surrogate and at least one misligation product surrogate; and so forth. The term “misligation” is generally intended to refer to products, surrogates, and the like that result from mismatch ligation reaction, but not match ligation reactions.

The term “ligation product surrogate” as used herein refers to any molecule or moiety whose detection or identification indicates the existence of one or more corresponding ligation products. A ligation product surrogate can, but need not, include at least part of the corresponding ligation product. Exemplary ligation product surrogates include but are not limited to, digested ligation products; amplified ligation products; digested amplified ligation products; one or more moieties cleaved or released from a ligation product or ligation product surrogate; one or more complementary strand or counterpart of a ligation product or a ligation product surrogate; reporter probes; including but not limited to cleavage and amplification products thereof; hybridization tag complements, including but not limited to ZipChutes™ (typically a molecule or complex comprising at least one hybridization tag complement, at least one mobility modifier, and at least one reporter group, generally a fluorescent reporter group; Applied Biosystems, see, e.g., Applied Biosystems Part Number 4344467 Rev. C; see also U.S. Provisional Patent Application Ser. No. 60/517470); and the like. The term “digested amplified ligation product” is intended to include a ligation product that is digested then amplified as well as a ligation product that is amplified then digested. Likewise, the terms “misligation product surrogate” and “(mis)ligation product surrogate” have the meanings in reference to misligation products or (mis)ligation products respectively.

As used herein, “ligation rate” or “rate” are relative terms that are determined by evaluating at least one measurable parameter of at least one (mis)ligation product generated by a given ligase. In certain embodiments, a “ligation rate ratio” or “ratio” is obtained by comparing at least one quantifiable parameter of at least one (mis)ligation product generated by a first ligase with the same measurable parameter of the (mis)ligation product generated by a given second ligase under the same conditions, i.e., ligase 1/ligase 2, or vice versa, as appropriate. By way of illustration, without limitation, if the integrated area under the curve corresponding to exemplary (mis)ligation product A obtained using Afu ligase, as determined with an AB PRISM® 3100 Genetic Analyzer using GeneScan® Analysis Software (Applied Biosystems), is 10 and the integrated area under the curve corresponding to exemplary (mis)ligation product B obtained using Taq ligase under the same conditions is 1, the corresponding ligation rate ratio is 10:1 (Afu:Taq) or 1:10 (Taq:Afu). In certain embodiments, the ligation rate for a given ligation product is compared to at least one corresponding standard curve. Those in the art appreciate that numerous measurable parameters exist that can be used to compare the quantity of (mis)ligation product generated by two ligases, including without limitation, (mis)ligation product peak height, integrated area under the curve for the (mis)ligation products, and so forth. By evaluating the ligation rate or the ligation rate ratio, one can identify and determine the quantity of at least one target nucleotide.

The term “mobility-dependent analytical technique” as used herein refers to any means for separating different molecular species based on differential rates of migration of those different molecular species in one or more separation processes. Exemplary mobility-dependent analytical techniques include electrophoresis, chromatography, mass spectroscopy, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682; PCT Publication No. WO 01/92579; D. R. Baker, Capillary Electrophoresis, Wiley-Interscience (1995); Biochromatography: Theory and Practice, M. A. Vijayalakshmi, ed., Taylor & Francis, London, U.K. (2003); Krylov and Dovichi, Anal. Chem. 72:111R-128R (2000); Swinney and Bornhop, Electrophoresis 21:1239-50 (2000); Crabtree et al., Electrophoresis 21:1329-35 (2000); and A. Pingoud et al., Biochemical Methods: A Concise Guide for Students and Researchers, Wiley-VCH Verlag GmbH, Weinheim, Germany (2002).

The term “mobility modifier” as used herein refers to at least one molecular entity, for example but not limited to, at least one polymer chain, that when added to at least one element (e.g., at least one probe, at least one primer, at least one ligation product, at least one ligation product surrogate, or combinations thereof) affects the mobility of the element to which it is hybridized or bound, covalently or non-covalently, in at least one mobility-dependent analytical technique. Typically, a mobility modifier changes the charge/translational frictional drag when hybridized or bound to the element; or imparts a distinctive mobility, for example but not limited to, a distinctive elution characteristic in a chromatographic separation medium or a distinctive electrophoretic mobility in a sieving matrix or non-sieving matrix, when hybridized or bound to the corresponding element; or both (see, e.g., U.S. Pat. Nos. 5,470,705 and 5,514,543; Grossman et al., Nucl. Acids Res. 22:4527-34, 1994). In certain embodiments, a multiplicity of probes exclusive of mobility modifiers, a multiplicity of primers exclusive of mobility modifiers, a multiplicity of ligation products exclusive of mobility modifiers, a multiplicity of ligation product surrogates exclusive of mobility modifiers, or combinations thereof, have the same or substantially the same mobility in at least one mobility-dependent analytical technique.

In certain embodiments, a multiplicity of probes, a multiplicity of primers, a multiplicity of ligation products, a multiplicity of ligation product surrogates, or combinations thereof, have substantially similar distinctive mobilities, for example but not limited to, when a multiplicity of elements comprising mobility modifiers have substantially similar distinctive mobilities so they can be bulk separated or they can be separated from other elements comprising mobility modifiers with different distinctive mobilities. In certain embodiments, a multiplicity of probes comprising mobility modifiers, a multiplicity of primers comprising mobility modifiers, a multiplicity of ligation products comprising mobility modifiers, a multiplicity of ligation product surrogates comprising mobility modifiers, or combinations thereof, have different distinctive mobilities.

In certain embodiments, at least one mobility modifier comprises at least one nucleotide polymer chain, including without limitation, at least one oligonucleotide polymer chain, at least one polynucleotide polymer chain, or both at least one oligonucleotide polymer chain and at least one polynucleotide polymer chain. For example but not limited to, a series of additional non-target sequence-specific nucleotides in one or more probes such as “TTTT” or [N]ₓ, where “N” is any nucleotide and “x” is integer corresponding to the number of the particular nucleotide that is repeated (see, e.g., Tables 2 and 5); or nucleotide spacers (see e.g., Tong et al., Nat. Biotech. 19:756-759, 2001). In certain embodiments, at least one mobility modifier comprises at least one non-nucleotide polymer chain. Exemplary non-nucleotide polymer chains include, without limitation, peptides, polypeptides, polyethylene oxide (PEO), or the like. In certain embodiments, at least one polymer chain comprises at least one substantially uncharged, water-soluble chain, such as a chain composed of one or more PEO units; a polypeptide chain; or combinations thereof.

The polymer chain can comprise a homopolymer, a random copolymer, a block copolymer, or combinations thereof. Furthermore, the polymer chain can have a linear architecture, a comb architecture, a branched architecture, a dendritic architecture (including without limitation, polymers containing polyamidoamine branched polymers, Polysciences, Inc. Warrington, Pa.), or combinations thereof. In certain embodiments, at least one polymer chain is hydrophilic, or at least sufficiently hydrophilic when hybridized or bound to an element to ensure that the element-mobility modifier is readily soluble in aqueous medium. Where the mobility-dependent analytical technique is electrophoresis, in certain embodiments, the polymer chains are uncharged or have a charge/subunit density that is substantially less than that of its corresponding element.

The synthesis of polymer chains useful as mobility modifiers will depend, at least in part, on the nature of the polymer. Methods for preparing suitable polymers generally follow well-known polymer subunit synthesis methods. These methods, which may involve coupling of defined-size, multi-subunit polymer units to one another, either directly or through charged or uncharged linking groups, are generally applicable to a wide variety of polymers, such as PEO, polyglycolic acid, polylactic acid, polyurethane polymers, polypeptides, oligosaccharides, and nucleotide polymers. Such methods of polymer unit coupling are also suitable for synthesizing selected-length copolymers, e.g., copolymers of PEO units alternating with polypropylene units. Polypeptides of selected lengths and amino acid composition, either homopolymer or mixed polymer, can be synthesized by standard solid-phase methods (see, e.g., Int. J. Peptide Protein Res., 35: 161-214, 1990).

One method for preparing PEO polymer chains having a selected number of hexaethylene oxide (HEO) units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (see, e.g., U.S. Pat. No. 4,914,210; see also, U.S. Pat. No. 5,777,096).

The term “nucleotide base”, as used herein, refers to a substituted or unsubstituted aromatic ring or rings. In certain embodiments, the aromatic ring or rings contain at least one nitrogen atom, such as a “nitrogenous base”. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 5 methyl-cytosine, uracil, thymine, and analogs of the naturally occurring nucleotide bases, including without limitation, 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7 mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different, —R, —OR, —NR₂ azide, cyanide or halogen groups, where each R is independently H, C₁-C₆ alkyl, C₂-C₇ acyl, or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352;, and WO 99/14226). Exemplary LNA sugar analogs within a polynucleotide include, but are not limited to, the structures: 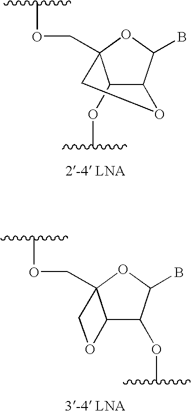
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, cyano, amido, imido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T, or U, the pentose sugar is attached to the N¹-position of the nucleotide base, except for pseudouridines, in which the pentose sugar is attached to the C⁵-position of the uracil nucleotide base (see, e.g., Kornberg and Baker, DNA Replication, 2^{nd} Ed., 1992, Freeman, San Francisco, Calif.).

One or more of the pentose carbons of a nucleotide may be substituted with a phosphate ester having the formula: 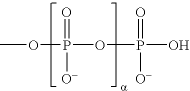
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. Review of nucleotide chemistry can be found in, among other places, Shabarova, Z. and Bogdanov, A. Advanced Organic Chemistry of Nucleic Acids, VCH, New York, 1994; and Nucleic Acids in Chemistry and Biology, Blackburn and Gait, eds., Oxford University Press, New York, N.Y.,1996 (hereinafter “Blackburn and Gait”).

The term “nucleotide analog”, as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers that can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone comprising at least one amide bond. See, e.g., Datar and Kim, Concepts in Applied Molecular Biology, Eaton Publishing, Westborough, Mass., 2003, particularly at pages 74-75; Verma and Eckstein, Ann. Rev. Biochem. 67:99-134, 1998; Goodchild, Bioconj. Chem., 1:165-187, 1990.

As used herein, the terms “polynucleotide”, “oligonucleotide”, “nucleic acid”, and “nucleic acid sequence” are generally used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, tetraalkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-40 when they are sometimes referred to in the art as oligonucleotides, to several thousands of monomeric nucleotide units. Nucleic acid sequence are shown in the 5′ to 3′ orientation from left to right, unless otherwise apparent from the context or expressly indicated differently; and in DNA, “A” denotes deoxyadenosine, “C” denotes deoxycytidine, “G” denotes deoxyguanosine, “T” denotes thymidine; while in RNA, “A” denotes adenosine, “C” denotes cytidine, “G” denotes guanosine, and “U” denotes uridine.

Nucleic acids include, but are not limited to, gDNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 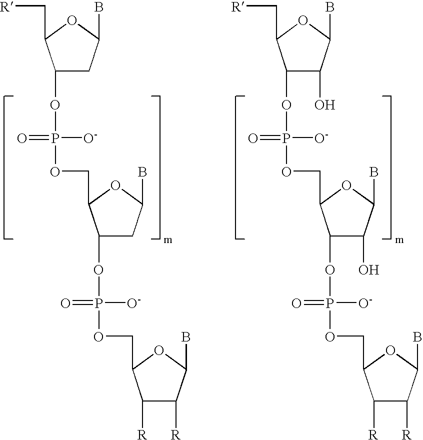
wherein each “B” is independently the base moiety of a nucleotide, e.g., a purine, a 7-deazapurine, a purine or purine analog substituted with one or more substituted hydrocarbons, a pyrimidine, a pyrimidine or pyrimidine analog substituted with one or more substituted hydrocarbons, or an analog nucleotide; each “m” defines the length of the respective nucleic acid and can range from zero to thousands, tens of thousands, or even more; each “R” is independently selected from the group comprising hydrogen, halogen, —R″, —OR″, and —NR″R″, where each R″ is independently (C1-C6)alkyl, (C2-C7)acyl or (C5-C14)aryl, cyanide, azide, or two adjacent Rs are taken together to form a bond such that the ribose sugar is 2′,3′-didehydroribose; and each “R′” is independently hydroxyl or 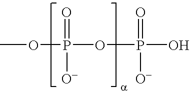
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “nucleic acid sequence”, “polynucleotide”, and “oligonucleotide” can also include nucleic acid analogs, polynucleotide analogs, and oligonucleotide analogs. The terms “nucleic acid analog”, “polynucleotide analog” and “oligonucleotide analog” are generally used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254:1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. See also, Scheit, Nucleotide Analogs, John Wiley, New York, (1980); Englisch, Agnew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1999.

The term “polymerase” is used in a broad sense herein and includes amplifying means such as DNA polymerases, enzymes that typically synthesize DNA by incorporating deoxyribonucleotide triphosphates or analogs in the 5′→3′ direction in a template-dependent and primer-dependent manner; RNA polymerases, enzymes that typically synthesize RNA by incorporating ribonucleotide triphosphates or analogs, generally in a template-dependent manner; and reverse transcriptases, also known as RNA-dependent DNA polymerases, that synthesize DNA by incorporating deoxyribonucleotide triphosphates or analogs in the 5′→3′ direction in primer-dependent manner, typically using an RNA template. Descriptions of polymerases can be found in, among other places, R. M. Twyman, Advanced Molecular Biology, Bios Scientific Publishers Ltd. (1999); Polymerase Enzyme Resource Guide, Promega, Madison, Wis. (1998); P. C. Turner et al., Instant Notes in Molecular Biology, Bios Scientific Publishers Ltd. (1997); and B. D. Hames et al., Instant Notes in Biochemistry, Bios Scientific Publishers Ltd. (1997).

The term “primer” as used herein refers to an oligonucleotide comprising at least one region that is complementary or substantially complementary to the primer-binding portion of at least one probe, at least one (mis)ligation product, at least one (mis)ligation product surrogate, or combinations thereof, including sequences that are complementary to any of these, and that can anneal with such primer-binding portions or their complement under appropriate conditions. Primers typically serve as initiation sites for certain amplification techniques, including but not limited to, primer extension and PCR. A primer that hybridizes with a multiplicity of different probe species, (mis)ligation product species, (mis)ligation product surrogate species, or combinations thereof, is referred to as a “universal primer”. In certain embodiments, at least one primer comprises at least one additional component, including but not limited to, at least one primer-binding portion, at least one reporter probe binding portion, at least one reporter group, at least one hybridization tag, at least one mobility modifier, at least one affinity tag, or combinations thereof.

The criteria for designing sequence-specific primers and probes are well known to persons of ordinary skill in the art. Detailed descriptions of probe and primer design that provide for sequence-specific annealing can be found in, among other places, Diffenbach and Dveksler, PCR Primer, A Laboratory Manual, Cold Spring Harbor Press, 1995; Blackburn and Gait; and Kwok et al., Nucl. Acids Res. 18:999-1005, 1990; as well as numerous probe and primer design software programs.

The term “probe” as used herein, refers to an oligonucleotide comprising a target-specific portion that is capable, under appropriate conditions, of hybridizing with at least a part of at least one corresponding target nucleic acid sequence, including without limitation at least one amplified target nucleic acid sequence or the complement of at least one target nucleic acid sequence. A probe may include Watson-Crick bases, analogs, or modified bases, including but not limited to, the AEGIS bases (from Eragen Biosciences), described, e.g., in U.S. Pat. Nos. 5,432,272; 5,965,364; and 6,001,983. Additionally, bases may be joined by a natural phosphodiester bond or a different chemical linkage. Different chemical linkages include, but are not limited to, at least one amide linkage or at least one Locked Nucleic Acid (LNA) linkage, described in, e.g., published PCT Application Nos. WO 00/56748 and WO 00/66604.

Probes typically are part of at least one ligation probe set comprising at least one first probe and at least one second probe, that typically can be ligated or misligated, depending on the circumstances. In certain embodiments, at least one probe comprises at least one mismatched nucleotide relative to at least one portion of its corresponding target nucleic acid sequence. In certain embodiments, at least one mismatched nucleotide is at the 3′-end of the upstream probe, the 5′-end of the downstream probe, or both. In certain embodiments, at least one probe further comprises at least one reporter group, at least one affinity tag, at least one hybridization tag, at least one mobility modifier, at least one primer-binding portion, at least one reporter probe-binding portion, or combinations thereof. The target-specific portions and, when present, the primer-binding and/or reporter probe-binding portions, of the probes are of sufficient length to permit specific annealing with complementary sequences in the template, primers, or reporter probes, as appropriate.

As used herein, a “probe set” comprises at least one first probe and at least one corresponding second probe that are designed to hybridize with the corresponding template. In a typical probe set there is a single species of first probe and at least two species of second probe, or vice versa. For example, certain OLA protocols utilize a single specie of downstream ligation probe (sometimes referred to as a locus specific oligonucleotide (LSO), a reporter probe, or a common probe) and at least two species of upstream ligation probe (sometimes referred to as allele specific oligonucleotides (ASOs) or wild type and mutant probes/primers) for each SNP or target nucleotide being interrogated (see, e.g., Landegren et al., Science 241:1077-80, 1988; Edelstein et al., J. Clin. Micro. 36(2):569-72, 1998; Shi, Clin. Chem. 47(2): 164-72, 2001). When the first probe and a second probe from a probe set are hybridized to the corresponding template, under appropriate conditions and in the presence or an appropriate ligase, the two probes are ligated together to form a ligation product, provided that they comprise appropriate reactive groups, for example without limitation, a free 3′ hydroxyl group on the upstream probe and a 5′ phosphate group on the downstream probe. In certain embodiments, the first and a corresponding second probe hybridize adjacently, while in other embodiments, after the probes hybridize an intervening gap-filling step renders the 3′-end of the upstream probe adjacent to the 5-end of the downstream probe.

In certain embodiments, the first probes and second probes in a probe set are designed with similar melting temperatures (Tₘ). Where a probe includes a complement of the target nucleotide being interrogated (sometimes referred to as a “pivotal complement”), preferably, the Tₘ for the probe(s) comprising the complement(s) of the target nucleotide will be approximately 4-6° C. lower than the other probe(s) in the probe set that do not contain the target nucleotide complement. The probe comprising the pivotal complement will also preferably be designed with a Tₘ near the ligation temperature. Thus, a probe with a mismatched nucleotide will more readily dissociate from the target at the ligation temperature. The ligation temperature, therefore, provides another way to discriminate between, for example, multiple potential alleles at a SNP site or alternative target nucleotides.

A mismatched base at the pivotal complement, however, may interfere with ligation since, absent misligation, it can't base pair with the SNP site nucleotide or the target nucleotide, even if both probes are otherwise fully hybridized to their respective target regions. Thus, highly related sequences that differ by as little as a single nucleotide can be distinguished, for example but not limited to, the alternative alleles at a SNP site, a single nucleotide mutation in a tumor repressor gene, such as p53, or a drug resistance mutation in a drug-resistant mutant microorganism or virus, such a point mutation in HIV protease from a patient being treated with one or more protease inhibitors, provided that misligation products are either not generated in detectable levels or do not become problematic. In certain embodiments, the pivotal complement is present at the 3′-end of the upstream probe, the 5′-end of the downstream probe, or both. In certain embodiments, the pivotal complement is not at the end of either the upstream probe or the downstream probe. For example without limitation, the pivotal complement can be the 3′ penultimate nucleotide on the upstream probe.

Those in the art understand that probes and probe sets that are suitable for use with the disclosed methods and kits can be identified empirically using the current teachings and routine methods known in the art, without undue experimentation. For example, suitable probes and probe sets can be obtained by selecting appropriate target nucleotides and target nucleotide sequences by searching relevant scientific literature, including but not limited to appropriate databases, that list or identify known SNPs, mutations, including but not limited to drug-induced mutations, chromosomal translocations, and the like; or by experimental analysis. When target nucleic acid sequences of interest are identified, test probes can be synthesized and modified if desired, using well known oligonucleotide synthesis and organic chemistry techniques (see, e.g., Current Protocols in Nucleic Acid Chemistry, Beaucage et al., eds., John Wiley & Sons, New York, N.Y., including updates through April 2004 (hereinafter “Beaucage et al.”); Blackburn and Gait; Glen Research 2002 Catalog; Sterling, Va.; and Synthetic Medicinal Chemistry 2003/2004, Berry and Associates, Dexter, Mich.). Test probes and/or probe sets are employed in the disclosed assays using appropriate target sequences and their suitability for interrogating the target nucleotide is evaluated. Standard curves can be generated, if desired, using pre-determined mixtures or serial dilutions of synthetic templates or gDNA as the target nucleic acid sequences in one or more of the disclosed ligation assays under standard conditions, as well known in the art (see, e.g., Overholtzer et al., Proc. Natl. Acad. Sci. 100:11547-52, 2002).

The term “reporter group” is used in a broad sense herein and refers to any identifiable tag, label, or moiety. The skilled artisan will appreciate that many different species of reporter groups can be used in the present teachings, either individually or in combination with one or more different reporter group. Exemplary reporter groups include, but are not limited to, fluorophores, radioisotopes, chromogens, enzymes, antigens including but not limited to epitope tags, heavy metals, dyes, phosphorescence groups, chemiluminescent groups, electrochemical detection moieties, affinity tags, binding proteins, phosphors, rare earth chelates, near-infrared dyes, including but not limited to, “Cy.7.SPh.NCS,” “Cy.7.OphEt.NCS,” “Cy7.OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Nebr.), electrochemiluminescence labels, including but not limited to, tris(bipyridal)ruthenium (II), also known as Ru(bpy)₃²⁺, Os(1,10-phenanthroline)₂bis(diphenylphosphino)ethane²⁺, also known as Os(phen)₂(dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal)ruthenium (II), also known as Ru(v-bpy₃²⁺), and the like.

The term reporter group also includes at least one element of multi-element reporter systems, e.g., affinity tags such as biotin/avidin, antibody/antigen, ligand/receptor including but not limited to binding proteins and their ligands, enzyme/substrate, and the like, in which one element interacts with other elements of the system in order to effect the potential for a detectable signal. Exemplary multi-element reporter systems include an oligonucleotide comprising at least one biotin reporter group and a streptavidin-conjugated fluorophore, or vice versa; an oligonucleotide comprising at least one dinitrophenyl (DNP) reporter group and a fluorophore-labeled anti-DNP antibody; and the like. In certain embodiments, reporter groups, particularly multi-element reporter groups, are not necessarily used for detection, but rather serve as affinity tags for separating, for example but not limited to, a biotin reporter group and a streptavidin coated substrate, or vice versa; a digoxygenin reporter group and an anti-digoxygenin antibody or a digoxygenin-binding aptamer; a DNP reporter group and an anti-DNP antibody or a DNP-binding aptamer; and the like. Detailed protocols for attaching reporter groups to oligonucleotides, polynucleotides, peptides, proteins, mono-, di- and oligosaccharides, organic molecules, and the like can be found in, among other places, Bioconjugate Techniques; Beaucage et al.; Molecular Probes Handbook; and Pierce Applications Handbook and Catalog 2003-2004, Pierce Biotechnology, Rockford, Ill. (2003; hereinafter Pierce Applications Handbook).

In certain embodiments, at least one reporter group comprises at least one electrochemiluminescent moiety that can, under appropriate conditions, emit detectable electrogenerated chemiluminescence (ECL). In ECL, excitation of the electrochemiluminescent moiety is electrochemically driven and the chemiluminescent emission can be optically detected. Exemplary electrochemiluminescent reporter group species include: Ru(bpy)₃²⁺ and Ru(v-bpy)₃²⁺ with emission wavelengths of 620 nm; Os(phen)₂(dppene)²⁺ with an emission wavelength of 584 nm; luminol/hydrogen peroxide with an emission wavelength of 425 nm; Al(hydroxyquinoline-5-sulfonic acid) with an emission wavelength of 499 nm; and 9,10-diphenylanothracene-2-sulfonate with an emission wavelength of 428 nm; and the like. Modified forms of these three electrochemiluminescent reporter group species that are amenable to incorporation into probes and coded molecular tags are commercially available or can be synthesized without undue experimentation using techniques known in the art. For example, a Ru(bpy)₃²⁺ N-hydroxy succinimide ester for coupling to nucleic acid sequences through an amino linker group has been described (see, U.S. Pat. No. 6,048,687); and succinimide esters of Os(phen)₂(dppene)²⁺ and Al(HQS)₃³⁺ can be synthesized and attached to nucleic acid sequences using similar methods. The Ru(bpy)₃²⁺ electrochemiluminescent reporter group can be synthetically incorporated into nucleic acid sequences using commercially available ru-phosphoramidite (IGEN International, Inc., Gaithersburg, Md.).

Additionally other polyaromatic compounds and chelates of ruthenium, osmium, platinum, palladium, and other transition metals have shown electrochemiluminescent properties. Detailed descriptions of ECL and electrochemiluminescent moieties can be found in, among other places, A. Bard and L. Faulkner, Electrochemical Methods, John Wiley & Sons (2001); M. Collinson and M. Wightman, Anal. Chem. 65:2576 et seq. (1993); D. Brunce and M. Richter, Anal. Chem. 74:3157 et seq. (2002); A. Knight, Trends in Anal. Chem. 18:47 et seq. (1999); B. Muegge et al., Anal. Chem. 75:1102 et seq. (2003); H. Abrunda et al., J. Amer. Chem. Soc. 104:2641 et seq. (1982); K. Maness et al., J. Amer. Chem. Soc. 118:10609 et seq. (1996); M. Collinson and R. Wightman, Science 268:1883 et seq. (1995); and U.S. Pat. No. 6,479,233.

The term “sample” is used in a broad sense and is intended to include a variety of biological sources that contain nucleic acids. Exemplary biological samples include, but are not limited to, whole blood; red blood cells; white blood cells; buffy coat; swabs, including but not limited to buccal swabs, throat swabs, vaginal swabs, urethral swabs, cervical swabs, throat swabs, rectal swabs, lesion swabs, abcess swabs, nasopharyngeal swabs, and the like; urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; plasma; serum; pulmonary lavage; lung aspirates; and tissues, including but not limited to, liver, spleen, kidney, lung, intestine, brain, heart, muscle, pancreas, and the like. The skilled artisan will appreciate that lysates, extracts, or material obtained from any of the above exemplary biological samples are also within the scope of the current teachings. Tissue culture cells, including explanted material, primary cells, secondary cell lines, and the like, as well as lysates, extracts, or materials obtained from any cells, are also within the meaning of the term sample as used herein. Microorganisms and viruses that may be present on or in a sample are also within the scope of the current teachings.

The term “target nucleic acid sequence” as used herein refers to a specific nucleic acid oligomer, typically gDNA or a synthetic template, that contains one or more target nucleotides. A target nucleotide is that nucleotide in the target nucleic acid sequence that is interrogated by one or more probes of one or more probe sets to determine its identity. Thus, a first target nucleotide is present in the first target nucleic acid sequence, a second target nucleotide is present in the second target nucleic acid sequence, and so forth. Those in the art understand that a particular target nucleic acid sequence may, but need not, comprise more than one target nucleotide. While the target nucleic acid sequence is generally described as a single-stranded molecule, it is to be understood that double-stranded molecules that contain one or more target nucleotides are also considered target nucleic acid sequences. Target nucleic acid sequences can serve as hybridization templates on which corresponding ligation probes can anneal. The amplification product of all or part of a nucleic acid sequence can also typically serve as a hybridization template on which corresponding probes can anneal. Such amplified sequences are within the intended scope of the term target nucleic acid sequence, unless otherwise apparent from the context or expressly excluded.

Those in the art will appreciate that the complement of the disclosed probe, target, and primer sequences, or combinations thereof, may also be employed in the methods herein. For example without limitation, a gDNA sample comprises both the target nucleic acid sequence and its complement. Thus when a genomic sample is denatured, both the target nucleic acid sequence and its complement are present in the sample as single stranded sequences. The probes described herein or their complement will specifically hybridize to the appropriate sequence, either the target nucleic acid sequences or its complement.

### II. TECHNIQUES

A target nucleic acid sequence according to the present teachings may be derived from any living, or once living, organism, including but not limited to, prokaryotes, archaea, viruses, and eukaryotes. The target nucleic acid may originate from the nucleus, typically gDNA, or may be extranuclear, e.g., plasmid, mitochondrial (including without limitation mitochondrial SNPs), viral, etc. The skilled artisan appreciates that gDNA includes not only full length material, but also fragments generated by any number of means, for example but not limited to, enzyme digestion, sonication, shear force, and the like, and that all such material, whether full length or fragmented, represent forms of target nucleic acid sequences.

A target nucleic acid sequence can be either synthetic or naturally occurring. Target nucleic acid sequences can be synthesized using oligonucleotide synthesis methods, and where appropriate, phosphorylation methods that are well-known in the art. Detailed descriptions of such techniques can be found in, among other places, Beaucage et al.; and Blackburn and Gait. Automated DNA synthesizers useful for synthesizing target nucleic acid sequences, probes, and primers are commercially available from numerous sources, including for example, the Applied Biosystems DNA Synthesizer Models 381A, 391, 392, and 394 (Applied Biosystems, Foster City, Calif.). Target nucleic acid sequences can also be generated biosynthetically, using in vivo methodologies and/or in vitro methodologies that are well known in the art. Descriptions of such technologies can be found in, among other places, Sambrook et al.; and Ausbel et al.

A variety of methods are available for obtaining a naturally occurring target nucleic acid sequences for use with the current teachings. Purified or partially purified nucleic acid is commercially available from numerous sources, including among others, Coriell Cell Repositories, Coriell Institute for Medical Research, Camden, N.J.; and the American Type Culture Collection (ATCC), Manassas, Va. When the target nucleic acid sequence is obtained through isolation from a biological matrix, preferred isolation techniques include (1) organic extraction followed by ethanol precipitation, e.g., using a phenol/chloroform organic reagent (see, e.g., Sambrook et al.; Ausbel et al.), for example using an automated DNA extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (see, e.g., Miller et al., Nucl. Acids Res. 16(3): 9-10, 1988), such precipitation methods being typically referred to as “salting-out” methods. Optimally, each of the above isolation methods is preceded by an enzyme digestion step to help eliminate unwanted protein from the sample, e.g., digestion with proteinase K, or other like proteases; a detergent step, or both (see, e.g., U.S. patent application Publication 2002/0177139; and U.S. patent application Ser. No. 10/618493). Commercially available nucleic acid extraction systems include, among others, the ABI PRISM® 6100 Nucleic Acid PrepStation and the ABI PRISM® 6700 Nucleic Acid Automated Work Station; nucleic acid sample preparation reagents and kits are also commercially available, including without limitation, NucPrep™ Chemistry, BloodPrep™ Chemistry, the ABI PRISM® TransPrep System, and PrepMan™ Ultra Sample Preparation Reagent (all from Applied Biosystems).

Ligation according to the present teachings comprises any enzymatic or non-enzymatic means wherein an inter-nucleotide linkage is formed between the opposing ends of nucleic acid probes that are adjacently hybridized on a target nucleic acid sequence to generate a (mis)ligation product. In certain embodiments, ligation also comprises at least one gap-filling procedure, wherein the ends of the two probes are not adjacently hybridized initially but the 3′-end of the upstream probe is extended by one or more nucleotide until it is adjacent to the 5′-end of the downstream probe, typically by a polymerase (see, e.g., U.S. Pat. No. 6,004,826). Exemplary ligases include without limitation, T4 DNA ligase, T4 RNA ligase, Thermus thermophilus (Tth) ligase, Thermus aquaticus (Taq) DNA ligase, Thermus scotoductus (Tsc) ligase, TS2126 (a thermophilic phage that infects Tsc) RNA ligase, Archaeoglobus flugidus (Afu) ligase, Pyrococcus furiosus (Pfu) ligase, Thermococcus kodakaraensis KOD1 ligase (lig_{Tk}), Rhodothermus marinus (Rm) ligase, Methanobacterium thermoautotrophicum (Mth) ligase, Aquifex aeolicus (Aae) ligase, Aeropyrum pernix K1 (Ape) ligase, or the like, including but not limited to reversibly inactivated ligases (see, e.g., U.S. Pat. No. 5,773,258), and enzymatically active mutants or variants thereof.

Ligation generally comprises at least one cycle of ligation, i.e., the sequential procedures of: hybridizing the target-specific portions of a first probe and a corresponding second probe to their respective complementary regions on the corresponding target nucleic acid sequences; ligating the 3′ end of the upstream probe with the 5′ end of the downstream probe to generate a ligation product; and denaturing the nucleic acid duplex to release the ligation product from the ligation product:target nucleic acid sequence duplex. The ligation cycle may or may not be repeated, for example, without limitation, by thermocycling the ligation reaction to amplify the (mis)ligation product using ligation probes, e.g., LDR, as distinct from using primers and polymerase to generate amplified (mis)ligation products, e.g., LCR (see, e.g., Cao, Trends in Biotech, 22(1):38-44, 2004).

Also within the scope of the teachings are ligation techniques such as gap-filling ligation, including, without limitation, gap-filling OLA, LDR, and LCR, bridging oligonucleotide ligation, and correction ligation. Descriptions of these techniques can be found in, among other places, U.S. Pat. Nos. 5,185,243 and 6,004,826; published European Patent Applications EP 320308 and EP 439182; and PCT Publication Nos. WO 90/01069 and WO 01/57268.

When used in the context of the present teachings, “suitable for ligation” refers to at least one first probe and at least one corresponding second probe, wherein each probe comprises an appropriately reactive group, typically a free hydroxyl group on the 3′ end of the upstream probe and a free phosphate group on the 5′ end of the downstream probe. Under appropriate conditions, phosphodiester bond formation between the 3′-hydroxyl group and the adjacent 5′-phosphate group is catalyzed by a ligase and a (mis)ligation product is generated.

Amplification according to the present invention encompasses any means by which at least one target nucleic acid sequence, at least a part of at least one (mis)ligation product, at least part of at least one (mis)ligation product surrogate, or combinations thereof, is reproduced, typically in a template-dependent manner, including without limitation, a broad range of techniques for amplifying nucleic acid sequences, either linearly or exponentially (i.e., generating an amplified (mis)ligation product or generating an amplified digested (mis)ligation product). Exemplary means for performing an amplifying step include LCR, PCR, primer extension, strand displacement amplification (SDA), multiple displacement amplification (MDA), nucleic acid strand-based amplification (NASBA), rolling circle amplification (RCA), transcription-mediated amplification (TMA), and the like, including multiplex versions or combinations thereof. Descriptions of such techniques can be found in, among other places, Sambrook and Russell; Sambrook et al.; Ausbel et al.; PCR Primer: A Laboratory Manual, Diffenbach, Ed., Cold Spring Harbor Press (1995); The Electronic Protocol Book, Chang Bioscience (2002)(“The Electronic Protocol Book”); Msuih et al., J. Clin. Micro. 34:501-07 (1996); The Nucleic Acid Protocols Handbook, R. Rapley, ed., Humana Press, Totowa, N.J., 2002(hereinafter “Rapley”); U.S. Pat. No. 6,027,998; PCT Publication Nos. WO 97/31256 and WO 01/92579; Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); and Schweitzer and Kingsmore, Curr. Opin. Biotechnol. 12:21-7 (2001).

In certain embodiments, amplification comprises at least one cycle of the sequential procedures of: hybridizing at least one primer with complementary or substantially complementary sequences in at least one target nucleic acid sequence, at least one (mis)ligation product, at least part of at least one (mis)ligation product, at least one (mis)ligation product surrogate, at least part of at least one (mis)ligation product surrogate, or combinations thereof; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally. In certain embodiments, newly-formed nucleic acid duplexes are not initially denatured, but are used in their double-stranded form in one or more subsequent steps.

Primer extension is an amplifying technique that comprises elongating at least one probe or at least one primer that is annealed to a template in the 5′→3′ direction using an amplifying means such as a polymerase. According to certain embodiments, with appropriate buffers, salts, pH, temperature, and nucleotide triphosphates, including analogs thereof, i.e., under appropriate conditions, a polymerase incorporates nucleotides complementary to the template strand starting at the 3′-end of an annealed probe or primer, to generate a complementary strand. In certain embodiments, primer extension can be used to fill a gap between two probes of a probe set that are hybridized to target sequences of at least one target nucleic acid sequence so that the two probes can be ligated together. In certain embodiments, the polymerase used for primer extension lacks or substantially lacks 5′ exonuclease activity.

The term “quantitative PCR”, or “Q-PCR” refers to a variety of well-known methods used to quantify the results of the polymerase chain reaction for specific nucleic acid sequences. Such methods typically are categorized as kinetics-based systems, that generally determine or compare the amplification factor, such as determining the threshold cycle (Cₜ), or as co-amplification methods, that generally compare the amount of product generated from simultaneous amplification of target and standard templates. Many Q-PCR techniques comprise reporter probes, intercalating dyes, or both. For example but not limited to TaqMan® probes (Applied Biosystems), i-probes, probes, molecular beacons, Eclipse probes, scorpion primers, Lux™ primers, FRET primers, ethidium bromide, SYBR® Green I (Molecular Probes), and PicoGreen® (Molecular Probes).

Separating comprises any process that removes at least some unreacted components, at least some reagents, or both some unreacted components and some reagents from at least one (mis)ligation product, at least one (mis)ligation product surrogate, or combinations thereof. In certain embodiments, at least one (mis)ligation product or at least part of at least one (mis)ligation product, at least one (mis)ligation product surrogate or at least part of at least one (mis)ligation product surrogate, or combinations thereof, are separated from unreacted components and reagents, including but not limited to, unreacted molecular species present in the sample, ligation reagents, digestion reagents, amplification reagents, for example, but not limited to, unbound/unhybridized ligation probes, primers, enzymes, co-factors, unbound sample components, nucleotides, and the like. The skilled artisan will appreciate that a number of well-known separation means can be useful with the methods disclosed herein.

Exemplary means for performing a separation step include gel electrophoresis, including but not limited to isoelectric focusing and capillary electrophoresis; dielectrophoresis; sorting, including but not limited to fluorescence-activated sorting techniques, such as flow cytometry; chromatography, including but not limited to HPLC, FPLC, size exclusion (gel filtration) chromatography, affinity chromatography, ion exchange chromatography, hydrophobic interaction chromatography, immunoaffinity chromatography, and reverse phase chromatography; affinity tag binding, such as biotin-avidin, biotin-streptavidin, maltose-maltose binding protein (MBP), and calcium-calcium binding peptide; aptamer-target binding; hybridization tag-hybridization tag complement annealing; spectroscopy, including but not limited to MALDI-TOF; and the like. In certain embodiments, at least one (mis)ligation product, at least one (mis)ligation product surrogate, or combinations thereof are bound to one or more substrates and separated from unbound components. Detailed discussion of separation techniques can be found in, among other places, Rapley; Sambrook et al.; Sambrook and Russell; Ausbel et al.; Molecular Probes Handbook; Pierce Applications Handbook; Capillary Electrophoresis: Theory and Practice, P. Grossman and J. Colburn, eds., Academic Press (1992); Wenz and Schroth, PCT International Publication No. WO 01/92579; and M. Ladisch, Bioseparations Engineering: Principles, Practice, and Economics, John Wiley & Sons (2001).

In certain embodiments, at least one separating step comprises at least one mobility-dependent analytical technique, for example but not limited to capillary electrophoresis. In certain embodiments, at least one separating step comprises at least one substrate, for example but not limited to binding at least one biotinylated nucleic acid molecule to at least one streptavidin-coated substrate. Suitable substrates include but are not limited to microarrays, appropriately treated or coated reaction vessels and surfaces, beads, for example but not limited to magnetic beads, latex beads, metallic beads, polymer beads, microbeads, and the like (see, e.g., Tong et al., Nat. Biotech. 19:756-59, 2001; Gerry et al., J. Mol. Biol. 292:251-62, 1999; Srisawat et al., Nucl. Acids Res. 29:e4, 2001; Han et al., Nat. Biotech. 19:631-35, 2001; Stears et al., Nat. Med. 9:140-45, including supplements, 2003). Those in the art will appreciate that the shape and composition of the substrate is generally not limiting. In certain embodiments, a plurality of (mis)ligation products or at least parts (mis)ligation products, (mis)ligation product surrogates or at least parts of (mis)ligation product surrogates, or combinations, thereof are separated via a mobility-dependent analysis technique.

In certain embodiments, at least one (mis)ligation product, at least one (mis)ligation product surrogate, or combinations thereof, are resolved (separated) by liquid chromatography. Exemplary stationary phase chromatography media for use in the teachings herein include reversed-phase media (e.g., C-18 or C-8 solid phases), ion-exchange media (particularly anion-exchange media), and hydrophobic interaction media. In certain embodiments, at least one (mis)ligation product, at least one (mis)ligation product surrogate, or combinations thereof can be separated by micellar electrokinetic capillary chromatography (MECC).

Reversed-phase chromatography is carried out using an isocratic, or more typically, a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. For separating polynucleotides, including (mis)ligation products and at least some (mis)ligation product surrogates, an ion-pairing agent (e.g., a tetra-alkylammonium) may be included in the solvent to mask the charge of phosphate.

The mobility of (mis)ligation products and at least some (mis)ligation product surrogates can be varied by using mobility modifiers comprising polymer chains that alter the affinity of the probe for the solid, or stationary phase. Thus, with reversed phase chromatography, an increased affinity of the (mis)ligation products and at least some (mis)ligation product surrogates for the stationary phase can be attained by adding a moderately hydrophobic tail (e.g., PEO-containing polymers, short polypeptides, and the like) to the mobility modifier. Longer tails impart greater affinity for the solid phase, and thus require higher non-polar solvent concentration for the ligation products and/or ligation product surrogates to be eluted (and a longer elution time).

In certain embodiments, at least one (mis)ligation product, at least one (mis)ligation product surrogate, or combinations thereof, are resolved by electrophoresis in a sieving or non-sieving matrix. In certain embodiments, the electrophoretic separation is carried out in a capillary tube by capillary electrophoresis (see, e.g., Capillary Electrophoresis: Theory and Practice, Grossman and Colburn eds., Academic Press, 1992). Exemplary sieving matrices for use in the disclosed teachings include covalently crosslinked matrices, such as polyacrylamide covalently crosslinked with bis-acrylamide; gel matrices formed with linear polymers (see, e.g., U.S. Pat. No. 5,552,028); and gel-free sieving media (see, e.g., U.S. Pat. No. 5,624,800; Hubert and Slater, Electrophoresis, 16: 2137-2142, 1995; Mayer et al., Analytical Chemistry, 66(10): 1777-1780, 1994). The electrophoresis medium may contain a nucleic acid denaturant, such as 7M formamide, for maintaining polynucleotides in single stranded form. Suitable capillary electrophoresis instrumentation are commercially available, e.g., the ABI PRISM™ Genetic Analyzer series (Applied Biosystems).

In certain embodiments, at least one hybridization tag complement includes at least one hybridization enhancer, where, as used herein, the term “hybridization enhancer” means moieties that serve to enhance, stabilize, or otherwise positively influence hybridization between two polynucleotides, e.g. intercalators (see, e.g., U.S. Pat. No.4,835,263), minor-groove binders (see, e.g., U.S. Pat. No. 5,801,155), and cross-linking functional groups. The hybridization enhancer may be attached to any portion of a mobility modifier, so long as it is attached to the mobility modifier is such a way as to allow interaction with the hybridization tag-hybridization tag complement duplex. In certain embodiments, at least one hybridization enhancer comprises at least one minor-groove binder, including without limitation, netropsin, distamycin, and the like.

The skilled artisan will appreciate that at least one (mis)ligation product, at least one (mis)ligation product surrogate, or combinations thereof, can also be separated based on molecular weight and length or mobility by, for example, but without limitation, gel filtration, mass spectroscopy, or HPLC, and detected using appropriate methods. In certain embodiments, at least one (mis)ligation product, at least one (mis)ligation product surrogate, or combinations thereof, are separated using at least one of the following forces: gravity, electrical, centrifugal, hydraulic, pneumatic, or magnetism.

In certain embodiments, at least one affinity tag is used to separate the element (e.g., a ligation product, a misligation product, a ligation product surrogate, a misligation product surrogate, etc.) to which it is bound from at least one component of a ligation reaction composition, a digestion reaction composition, an amplified ligation reaction composition, or the like. In certain embodiments, at least one affinity tag is used to bind at least one ligation product, at least one misligation product, at least one ligation product surrogate, at least one misligation product surrogate, or combinations thereof, to at least one substrate, for example but not limited to at least one biotinylated (mis)ligation product to at least one substrate comprising streptavidin. In certain embodiments, at least one aptamer is used to bind at least one ligation product, at least one misligation product, at least one ligation product surrogate, at least one misligation product surrogate, or combinations thereof, to at least one substrate (see, e.g., Srisawat and Engelke, RNA 7:632-641, 2001; Holeman et al., Fold Des. 3:423-31, 1998; Srisawat et al., Nucl. Acid Res. 29(2):e4, 2001).

In certain embodiments, at least one hybridization tag, at least one hybridization tag complement, or at least one hybridization tag and at least one hybridization tag complement, is used to separate the element to which it is bound or annealed from at least one component of a ligation reaction composition, a digestion reaction composition, an amplified ligation reaction composition, or the like. In certain embodiments, hybridization tags are used to attach at least one ligation product, at least one misligation product, at least one misligation product surrogate, at least one ligation product surrogate, or combinations thereof, to at least one substrate. In certain embodiments, at least two (mis)ligation products, at least two (mis)ligation product surrogates, or at least one (mis)ligation product and at least one (mis)ligation product surrogate, comprise the same hybridization tag. In certain embodiments, such a “universal” hybridization tag can then be used for bulk separation, including without limitation, separating a multiplicity of different element:hybridization tag species using the same hybridization tag complement; tethering a multiplicity of different element:hybridization tag species to a substrate comprising the same hybridization tag complement; or both.

In certain embodiments, different ligation products, different misligation products, different ligation product surrogates, different misligation product surrogates, or combinations thereof, are detected by mobility discrimination using separation techniques such as electrophoresis, mass spectroscopy, or chromatography rather than hybridization to capture oligonucleotides on a support. In these embodiments the probes may comprise hybridization tags of unique identifiable lengths or molecular weights. Alternatively, in certain embodiments, a hybridization tag portion of at least one probe is complementary to a particular mobility-modifier comprising a hybridization tag complement for selectively binding to the hybridization tag of the corresponding (mis)ligation product or (mis)ligation product surrogates, and a tail for effecting a particular mobility in a mobility-dependent analytical technique, including without limitation, electrophoresis (see, e.g., U.S. patent application Ser. No. 09/522,640). Thus, the ligation products, misligation products, ligation product surrogates, misligation product surrogates, or combinations thereof, can be separated by molecular weight or length to distinguish the individual sequences. The detection of a (mis)ligation product or a (mis)ligation product surrogate in a particular molecular weight or length bin indicates the presence of the corresponding target sequence in the sample. Descriptions of mobility discrimination techniques may be found, among other places, in U.S. Pat. Nos. 5,470,705; 5,514,543; 5,580,732; 5,624,800; and 5,807,682.

In an exemplary protocol, a variety (mis)ligation products of uniquely identifiable molecular mobility generated from a multiplex OLA or LDR reaction, are diluted in deionized formamide or other suitable diluent. The diluted (mis)ligation products are combined with an electrophoretic size standard (e.g., GS 500 or LIZ 120 size standards, Applied Biosystems) and loaded onto an electrophoresis platform (e.g., ABI Prism™ 3100 Genetic Analyzer, Applied Biosystems) and electrophoresed in POP-4 polymer at 15 kV using a 50 μL capillary. The bands are detected and their position relative to the marker is determined and plotted on an electropherogram. The bands are identified based on their relative electrophoretic mobility, indicating the presence of their corresponding target sequence in the sample.

In certain embodiments, at least one ligation product, at least one misligation product, at least one ligation product surrogate, at least one misligation product surrogate, or combinations thereof, comprises at least one hybridization tag containing a sequence that is complementary to a mobility-modifier comprising a hybridization tag complement that corresponds to the hybridization tag portion of at least one ligation product, at least one misligation product, at least one ligation product surrogate, at least one misligation product surrogate, or combinations thereof, and a tail, for effecting a particular mobility in a mobility-dependent analytical technique, such that when the hybridization tag portion and the mobility modifier comprising the corresponding hybridization tag complement are annealed a stable complex is formed (see, e.g., U.S. patent application Ser. No. 09/522,640).

According to certain of these embodiments, hybridization tag portions and hybridization tag complements should form a complex that (1) is stable under conditions typically used in nucleic acid analysis methods, e.g., aqueous, buffered solutions at room temperature; (2) is stable under mild nucleic-acid denaturing conditions; and (3) does not adversely effect a sequence specific binding of a target-specific portion of a probe with a target nucleic acid sequence. In addition, hybridization tag portions and hybridization complements should accommodate sets of distinguishable hybridization tag portions and hybridization tag complements such that a plurality of different (mis)ligation products, (mis)ligation product surrogates, or combinations thereof, and associated mobility modifiers may be present in the same reaction volume without causing cross-interactions among the hybridization tag portions, hybridization tag complements, target nucleic acid sequence and target-specific portions of the probes. Methods for selecting sets of hybridization tag sequences that minimally cross hybridize are described elsewhere (see, e.g., Brenner and Albrecht, PCT Patent Application No. WO 96/41011).

In certain embodiments, at least one hybridization tag and at least one corresponding hybridization tag complement pair includes a hybridization tag comprising conventional synthetic polynucleotide, and a hybridization tag complement that is PNA. PNA and PNA/DNA chimera molecules can be synthesized using well-known methods on commercially available, automated synthesizers, with commercially available reagents (see, e.g., Dueholm, et al., J. Org. Chem., 59:5767-73, 1994; Vinayak, et al., Nucleosides & Nucleotides, 16:1653-56, 1997).

The hybridization tag portions of certain probes may comprise all, part, or none of the target-specific portion of the probe; likewise, the hybridization tag portions of certain primers may comprise all, part, or none of the complement of the primer-binding portion of corresponding probes. In other embodiments, the hybridization tag portions of certain probes and/or certain primers do not comprise any portion of the target-specific portion of the probe or the complement of the primer-binding portion, respectively.

In certain embodiments, mobility modifiers comprise a hybridization tag complement portion for binding to the corresponding hybridization tag complement of (mis)ligation products, (mis)ligation product surrogates, or combinations thereof, and a tail for effecting a particular mobility in a mobility-dependent analytical technique.

In certain embodiments, the tail portion of a mobility modifier may be any entity capable of affecting a particular mobility of a amplification product/mobility-modifier complex in a mobility-dependent analysis technique. In certain embodiments, the tail portion of the mobility modifier should (1) have a low polydispersity in order to effect a well-defined and easily resolved mobility, e.g., Mw/Mn less than 1.05; (2) be soluble in an aqueous medium; (3) not adversely affect probe-target hybridization or addressable support-specific portion/tag complement binding; and (4) be available in sets such that members of different sets impart distinguishable mobilities to their associated complexes.

In certain embodiments of the teachings herein, the tail portion of the mobility modifier comprises a polymer. Specifically, the polymer forming the tail may be homopolymer, random copolymer, or block copolymer. Furthermore, the polymer may have a linear, comb, branched, or dendritic architecture. In addition, although the invention is described herein with respect to a single polymer chain attached to an associated mobility modifier at a single point, the invention also contemplates mobility modifiers comprising more than one polymer chain element, where the elements collectively form a tail portion.

Certain polymers for use herein are hydrophilic, or at least sufficiently hydrophilic when bound to a hybridization tag complement to ensure that the hybridization tag complement is readily soluble in aqueous medium. Where the mobility-dependent analytical technique is electrophoresis, the polymers are preferably uncharged or have a charge/subunit density that is substantially less than that of the (mis)ligation product or (mis)ligation product surrogate.

In certain embodiments, the polymer is polyethylene oxide (PEO), e.g., formed from one or more hexaethylene oxide (HEO) units, where the HEO units are joined end-to-end to form an unbroken chain of ethylene oxide subunits. Other exemplary embodiments include a chain composed of N 12mer PEO units, and a chain composed of N tetrapeptide units, where N is an adjustable integer (see, e.g., U.S. Pat. No. 5,777,096).

The synthesis of polymers useful as tail portions of a mobility modifier of the current teachings will depend on the nature of the polymer. Methods for preparing suitable polymers generally follow well known polymer subunit synthesis methods. Methods of forming selected-length PEO chains are discussed below. These methods, which involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, and oligosaccharides. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Fields and Noble, Int. J. Peptide Protein Res., 35: 161-214, 1990).

According to one method for preparing PEO polymer chains having a selected number of HEO units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., U.S. Pat. No. 4,914,210).

PNA is another polymer that can be used as a tail portion according to the current teachings. The advantages, properties and synthesis of PNA have been described above. In particular, when used in the context of a mobility-dependent analytical technique comprising an electrophoretic separation in free solution, PNA has the advantageous property of being essentially uncharged.

Coupling of the polymer tails to a hybridization tag complement can be carried out by an extension of conventional phosphoramidite polynucleotide synthesis methods, or by other standard coupling methods, e.g., a bis-urethane tolyl-linked polymer chain may be linked to an polynucleotide on a solid support via a phosphoramidite coupling. Alternatively, the polymer chain can be built up on a polynucleotide (or other tag portion) by stepwise addition of polymer-chain units to the polynucleotide, e.g., using standard solid-phase polymer synthesis methods.

As noted above, the tail portion of the mobility modifier imparts a mobility to a (mis)ligation product (surrogate):mobility modifier complex that is distinctive for each different complex. The contribution of the tail to the mobility of the complex will in generally depend on the size of the tail. However, addition of charged groups to the tail, e.g., charged linking groups in the PEO chain, or charged amino acids in a polypeptide chain, can also be used to achieve selected mobility characteristics in the probe/mobility modifier complex. Those in the art will also understand that the mobility of a complex may be influenced by the properties of the (mis)ligation product or (mis)ligation product surrogate, e.g., in electrophoresis using a sieving medium, a larger probe will reduce the electrophoretic mobility of the probe/mobility modifier complex.

In certain embodiments, the hybridization tag complement portion of a mobility modifier may be any entity capable of binding to, and forming a complex with, the corresponding hybridization tag of a ligation product, misligation product, ligation product surrogate, misligation product surrogate, or combinations thereof. Furthermore, the hybridization tag complement portion of the mobility modifier may be attached to the tail portion using conventional means.

When a hybridization tag complement is a polynucleotide, e.g., PNA, the hybridization tag complement may comprise all, part, or none of the tail portion of the mobility modifier. In some embodiments, the hybridization tag complement may consist of some or all of the tail portion of the mobility modifier. In other embodiments, the hybridization tag complement does not comprise any portion of the tail portion of the mobility modifier. For example without limitation, because PNA is uncharged, particularly when using free solution electrophoresis as the mobility-dependent analysis technique, the same PNA oligomer may act as both a hybridization tag complement and a tail portion of a mobility modifier.

According to certain embodiments, a plurality of (mis)ligation product (surrogate)/mobility modifier complexes are resolved via a mobility-dependent analytical technique.

In certain embodiments, identifying comprises detecting at least one reporter group that corresponds to at least one ligation product, at least one misligation product, at least one ligation product surrogate, at least one misligation product surrogate, or combinations thereof. According to the present teachings, identifying comprises detecting the presence or absence of a particular (mis)ligation product, a particular (mis)ligation product surrogate, or combinations thereof, particularly when hybridized to a support or occupying a particular mobility address during a mobility dependent analytical technique. For example, when the hybridization tag of a (mis)ligation product, (mis)ligation product surrogate, or their complement, specifically hybridizes to the hybridization tag complement on a substrate, the hybridized sequence can be detected provided that a reporter group is present. Typically, the reporter group provides an emission that is detectable or otherwise identifiable in the detection step. The type of detection process used will depend on the nature of the reporter group to be detected. For example without limitation, a particular detection step used in combination with a fluorescent reporter group, the fluorescent reporter group is detected using laser-excited fluorescent detection.

In certain embodiments, determining further comprises quantifying at least one detected (mis)ligation product, at least one detected (mis)ligation product surrogate, or combinations thereof, for example but not limited to graphically displaying the quantified at least one (mis)ligation product, at least one (mis)ligation product surrogate, or combinations thereof, on a graph, monitor, electronic screen, magnetic media, scanner print-out, or other two- or three-dimensional display. Typically the peak height, the area under the peak, the signal intensity of one or more detected reporter group on at least one (mis)ligation product, at least one (mis)ligation product surrogate, or combinations thereof, or other quantifiable parameter of the (mis)ligation product or (mis)ligation product surrogate are measured and the quantity of (mis)ligation product that was generated in a particular ligation assay can be inferred. Generally, at least one quantified parameter for at least one (mis)ligation product, at least one (mis)ligation product surrogate, or combinations thereof, generated by one ligase is compared to the same parameter(s) for the same (mis)ligation product, (mis)ligation product surrogate, or combinations thereof, generated by a second ligase and the ratio of the two (mis)ligation products is obtained.

In certain embodiments, at least one determining step comprises detecting and quantifying at least one (mis)ligation product parameter using at least one instrument, i.e., using an automated or semi-automated determining means that can, but need not, comprise a computer algorithm. In certain embodiments, the determining step is combined with or is a continuation of at least one separating step, for example but not limited to a capillary electrophoresis instrument comprising at least one fluorescent scanner and at least one graphing, recording, or readout component; a chromatography column coupled with an absorbance monitor or fluorescence scanner and a graph recorder; or a microarray with a data recording device such as a CCD camera. Exemplary means for performing a determining step include the ABI PRISM® 3100 Genetic Analyzer, ABI PRISM® 3100-Avant Genetic Analyzer, ABI PRISM® 3700 DNA Analyzer, ABI PRISM® 3730 DNA Analyzer, ABI PRISM® 3730x/DNA Analyzer (all from Applied Biosystems); the ABI PRISM® 7300 Real-Time PCR System; and microarrays and related software such as the ABI PRISM® 1700 (Applied Biosystems) and other commercially available array systems available from Affymetrix, Agilent, and Amersham Biosciences, among others (see also Gerry et al., J. Mol. Biol. 292:251-62, 1999); De Bellis et al., Minerva Biotec 14:247-52, 2002; and Stears et al., Nat. Med. 9:140-45, including supplements, 2003). Exemplary software includes GeneMapper™ Software, GeneScan® Analysis Software, and Genotyper® software (all from Applied Biosystems).

The generation of misligation products may be undesirable in certain ligation-based assays, depending on their application, while for other applications, the generation of misligation products may be desired. For example, conventional SNP genotyping typically is used to evaluate whether a given individual is homozygous or heterozygous at a particular SNP site. For illustration purposes, assume that SNP site 1 has two possible alleles T and G. Thus one expects the assay to identify the SNP as all T or all G if the individual is homozygous, or approximately half T and half G if the individual is heterozygous for SNP 1. Consequently, if the ligation assay results indicate, for example, 85% G (the match ligation product) and 15% T, due to mismatch ligation, the individual would likely be identified as a G homozygote at SNP 1. However, for certain ligation-based assay applications, such as analyzing nucleic acid sequences that from more than one individual, the presence of detectable misligation products could result in an incorrect determination.

According to the present teachings, at least one step for interrogating at least one target nucleotide is performed using the disclosed probes and probe sets; at least one step for generating at least one (mis)ligation product is performed using the disclosed ligation agents and ligation techniques; at least one step for generating at least one amplified (mis)ligation product and/or (mis)ligation product surrogate is performed using the disclosed amplifying means and amplification techniques; at least one step for generating at least one digested (mis)ligation product is performed using the disclosed nucleases, restriction enzymes, chemical digesting means, and digestion techniques; and at least one step for identifying at least one target nucleotide is performed using at least one disclosed detecting technique, at least one quantifying technique, at least one evaluating technique, at least one disclosed separating technique, or combinations thereof.

### III. EXEMPLARY EMBODIMENTS

The current teachings, having been described above, may be better understood by reference to examples. The following examples are intended for illustration purposes only, and should not be construed as limiting the scope of the teachings herein in any way.

The present teachings are directed to methods, reagents, and kits that are useful for generating at least one (mis)ligation product and/or identifying at least one target nucleotide. The skilled artisan will appreciate that when analyzing genomic DNA there are typically multiple copies of target nucleic acid sequences in the sample being evaluated, at least some of which contain the target nucleotide. The identity of, including the quantity or percentage of, a particular target nucleotide is generally determined from the sum of at least some of the ligation products obtained using at least part of that population of target nucleic acid sequences.

In certain embodiments, at least one target nucleotide is identified by comparing one or more quantified parameters between two or more (mis)ligation products or their surrogates, at least one quantified (mis)ligation product parameter and one or more standard curve, or both. In certain embodiments, at least one probe set comprises one or more nucleotides on or near the 3′-end of the upstream probe, on or near the 5′-end of the downstream probe, or both, that are not complementary to the corresponding nucleotide(s) on the target nucleic acid sequence. The corresponding nucleotide on the target nucleic acid sequence can, but need not, be the target nucleotide. In certain embodiments, the ligation site comprises the nucleotide opposing the target nucleotide. Those in the art will appreciate that the terms upstream or 5′ probe and downstream or 3′ probe are used in reference to their annealing position on the corresponding target nucleic acid sequence in the 3′→5′ orientation.

### EXAMPLE 1

### Cloning and Expression of Afu Ligase

Archaeoglobus fulgidus gDNA was purchased from ATCC (catalog #49558D). Two oligonucleotides derived from the sequence of the putative Afu ligase gene (GenBank seq. AF0623) were designed: a 5′-primer containing an Ndel site (in bold), 5′-CATATGATGTTGTTTGCCGAGTTTG-3′ (SEQ ID NO:1), and a 3′ primer containing a Sal I site (in bold), 5′-TCGACTCATTGTCTCTTTACCTCGAACTG-3′ (SEQ ID NO:2). The polymerase chain reaction (PCR) was performed under the following conditions: 1× Pfu buffer (Stratagene), 200 mM each dNTP (Pharmacia), 5 pmol each primer, 70 ng gDNA and 2.5 u of Pfu HotStart DNA polymerase (Stratagene) were combined in reaction composition with a final volume of 25 μL. Cycling conditions were: 95° C. for 2 minutes followed by 30 cycles (96° C. for 5 s, 60° C. for 30 s, 72° C. for 2 minutes) followed by 70° C. for 10 minutes. The PCR amplified fragment was gel-purified. To enable the use of the TOPO cloning vector (Invitrogen) the PCR fragment was incubated with Taq DNA polymerase for the non-templated addition of adenine. The resulting fragment was cloned into PCR 4 TOPO TA vector (Invitrogen) according to the manufacturer's protocol. The presence of the putative ligase gene was confirmed by DNA sequencing and the resulting plasmid was subcloned into the pET24a expression vector. E. coli CodonPlus BL21 (DE3) (Stratagene) was used for protein expression. Overnight cultures were grown in LB medium containing 50 μg/mL chloramphenicol and 50 μg/μL kanamycin. The next morning 1.5 L of LB medium without antibiotics was inoculated with 75 ml of the overnight culture and incubated at 37° C. with shaking at 250 rpm for 2 hours. Protein synthesis was induced by the addition of isopropyl-D-thiogalactopyranoside (IPTG) to a final concentration of 1 mM. After 2 hours of induction, cells were harvested (5,000×g, 20 min, 4° C.) and the cell pellets were stored at −80 ° C.

### EXAMPLE 2

### rAfu Ligase Purification

The cell pellets from Example 1 were re-suspended in buffer A at 1/10^{th} of the original volume (buffer A: 50 mM Tris-HCl [pH 7.5]). The cells were disrupted by sonication and the lysate clarified by centrifugation (12,000×g, 20 min, 4° C.). The soluble fraction of the lysate was pasteurized at 85° C. for 25 min followed by 10 minutes on ice. Precipitated E. coli proteins were removed by centrifugation (12,000×g, 20 min, 4° C.). An anion exchange column (Macro-Prep High Q, Bio-Rad, Hercules, Calif.) and a cation exchange column (Macro-Prep High S, Bio-Rad, Hercules, Calif.) were connected in series and both equilibrated with buffer A. The supernatant was applied first to the anion-exchange resin and the flow-through applied directly to the cation-exchange column. The recombinant Afu ligase (rAfu) did not bind to the anion exchange resin. The cation exchange column was washed with buffer A until the background returned to zero. The enzyme was eluted with a 0 to 1.0 M MgCl₂ linear gradient with buffer A. The rAfu eluted between 0.15 and 0.30 M MgCl₂. Peak fractions were analyzed by SDS-PAGE gel electrophoresis. Fractions containing rAfu were pooled, concentrated and desalted with an Amicon pressure concentrator cell (Millipore, Bedford, Mass.) and mixed 1:1 with storage buffer (90% glycerol in a buffer containing 20 mM Tris, pH 8.5, 0.1 mM EDTA, 0.05% Triton X-100, 0.05% Tween 20). Resulting purified rAfu ligase was stored at −20° C. The protein concentration was determined by the Bio-Rad protein assay system (Bio-Rad, Hercules, Calif.) using bovine serum albumin as a standard. DNA ligation activity was measured with the thermostable units assay as described for Taq DNA ligase (New England BioLabs, Beverly, Mass.), with 1 unit defined as the amount of enzyme that ligates 50% of bacteriophage λ cos-sites of 1 μg Hind III λ-DNA in 15 min at 45° C.

### EXAMPLE 3

### Oligonucleotide Synthesis

The synthetic oligonucleotides described herein, including templates, probes, and primers, were prepared as follows. Oligonucleotides were synthesized on an ABI PRISM® 380A DNA Synthesizer using N,N-diisopropylphosphoramidites, controlled-pore glass columns, and synthesis reagents (all from Applied Biosystems, Foster City, Calif.) using a 10-fold excess of protected phosphoramidites and 1 μmole of nucleotide bound to the synthesis support column according to the manufacturer's protocols. The 5′-end of all of the downstream (3′-) probes in the examples described herein were chemically phosphorylated to render them suitable for ligation. The synthesis was performed according to the manufacturer's instructions (see also, Matteucci, et al., Journal Amer. Chem. Soc., 103:3185-3319, 1981; McBride, et al., Tetrahedron Letters, 24:245-248, 1983). The repetitive yield of the synthesis, as measured by the optical density of the removed protecting group, was greater than 97.5%. The crude oligonucleotide mixtures were purified by preparative HPLC.

### EXAMPLE 4

### Ligase Fidelity Using Synthetic Templates

To assess the fidelity of Afu ligase relative to that of two Thermus species ligases, a series of synthetic templates (derived from the −21 primer of bacteriophage M13) and a corresponding ligation probe set were generated on an ABI PRISM® 380A DNA Synthesizer, as described in Example 3. The four M13-derived templates, each differing by a single nucleotide at the ligation site (underlined), are shown in Table 1.

TABLE 1
M13-Derived Templates

| | |
|---|---|
| Template | |
| No. | Sequence and SEQ ID NO: |

| | | |
|---|---|---|
| 1 | ACATTTTGCTGCCGGTCACGGTTCGAA | (SEQ ID NO.:1) |
| | CGTACGGACG | |
| 2 | ACATTTTGCTGCCGGTCTCGGTTCGAA | (SEQ ID NO.:2) |
| | CGTACGGACG | |
| 3 | ACATTTTGCTGCCGGTCGCGGTTCGAA | (SEQ ID NO.:3) |
| | CGTACGGACG | |
| 4 | ACATTTTGCTGCCGGTCCCGGTTCGAA | (SEQ ID NO.:4) |
| | CGTACGGACG | |

The probe set, derived from the −21M13 primer, comprised a single downstream probe and four upstream probes, as shown in Table 2. The single downstream probe, an LSO in this example, comprised the fluorescent reporter group FAM® (Fam). The four upstream probes each comprised the same template-specific sequence except for the 3′ nucleotide and three of the four further comprised additional T residues that served as mobility modifiers (shown in brackets).

TABLE 2

Probe Set derived from the
−21 primer of bacteriophage M13 (<21M13).

| | |
|---|---|
| Upstream Probes | Downstream Probe |

| | |
|---|---|
| TGTAAAACGACGGCCAGT | pGCCAAGCTTCGATGC |

| | |
|---|---|
| (SEQ ID NO:5; probe no. 5) | CTG C-Fam |
| | (SEQ ID NO:6) |

[TTTT]TGTAAAACGACGGCCAGA

(SEQ ID NO:7; probe no. 7)

[TTTTTTTT]TGTAAAACGACGGCCAGC

(SEQ ID NO:8; probe no. 8)

[TTTTTTTTTTTTT]TGTAAAACGACGGCCAGG

(SEQ ID NO:9; probe no. 9)

Individual ligation reaction compositions comprising: a ligase (either Taq ligase, AK16D ligase, or Afu ligase); one of the four templates shown in Table 1; and the ligation probe set shown in Table 2; were prepared as follows. A series of 10× DNA premixes for each template and corresponding ligation probe set were prepared comprising 400 nM of the common downstream probe, 200 nM of each of the four upstream probes, and 800 nM of one of the synthetic templates in appropriate ligation buffer (50 mM Tris-HCl pH 7.5, 10 mM MgCl₂, 10 mM DTT, 1 mM ATP, and 0.1% Triton X-100 for Afu ligase; 20 mM Tris-HCl pH 7.6, 25 mM KAc, 10 mM MgCl₂, 10 mM DTT, 1 mM NAD and 0.1% Triton X-100 for either of the Thermus ligases). Each DNA premix was heated to 90° C. then slowly cooled to 40° C. over ten minutes in the block of a Model 9600 Thermocycler (Perkin Elmer) using the ramp function, allowing hybridization complexes to form. Five μL of each premix comprising double-stranded hybridization complexes was combined with 45 μL of the ligase/buffer mixture (Taq ligase (New England BioLabs), AK16D ligase (prepared as described in Tong et al., Nucl. Acids Res. 27(3):788-94, 1999) or Afu ligase, each in appropriate ligation buffer). These ligation reaction compositions were placed in the thermocycler at 45° C.

Each reaction was stopped by combining a 5 μL aliquot of the cycled ligation reaction composition with 10 μL 100 mM EDTA and chilled on ice. For the matched ligation reactions, aliquots were obtained after 2 minutes (min.), 5 min., 10 min., 20 min., 30 min., and 60 min. reaction time; for the mismatched ligation reactions, aliquots were obtained after 10 min., 20 min., 30 min., 60 min., 4 hours (hr.), 10 hr., 21 hr. and 22 hr. reaction time. Each of these stopped reaction compositions was diluted 40-fold in distilled deionized water and a 5 μL aliquot of the diluted ligation products was combined with 20 μL HiDi™ formamide containing 0.2 μL LIZ™ 120 size standards. These diluted ligation products were separated by electrophoresis using 36 cm capillaries with POP4 polymer on an ABI PRISM® 3100 Genetic Analyzer and the resulting electropherograms were analyzed using GeneScan version 2.1 software (all from Applied Biosystems). Relative yield was calculated as the ratio of the peak area of the ligation product compared to the total peak areas of the ligation product and unreacted ligation probes. Under steady state conditions, i.e., relative yield <20%, ligation product formation was linear with time. Only data in the linear range was considered in this and other examples described herein, except for data from the zero time point. The mismatch ligation rates, normalized to the rate of matched ligation using the same upstream probe, are shown in Table 3.

TABLE 3
Relative rates of mismatch ligation for Taq ligase, AK16D ligase,
and Afu ligase using M-13 derived probes and templates.

| | Template | | | |
|---|---|---|---|---|
| probe no. | (target | | | |
| (3′ nucleotide) | nucleotide) | Taq ligase | AK16D ligase | Afu ligase |
| 9 (G) | 1 (A) | — | — | — |
| 9 (G) | 2 (T) | 0.03% | 0.04% | — |
| 9 (G) | 3 (G) | — | — | — |
| 5 (T) | 4 (C) | 0.07% | 0.04% | 1.51% |
| 5 (T) | 2 (T) | 0.01% | 0.03% | — |
| 5 (T) | 3 (G) | 0.25% | 0.22% | — |
| 7 (A) | 4 (C) | 0.05% | 0.05% | 1.68% |
| 7 (A) | 1 (A) | — | — | — |
| 7 (A) | 3 (G) | — | — | — |
| 8 (C) | 4 (C) | — | — | — |
| 8 (C) | 1 (A) | 0.04% | 0.06% | 0.92% |
| 8 (C) | 2 (T) | — | — | 0.33% |

“—” indicates no detectable misligation in all tables.

Comparing the experimentally determined mismatch ligation rates for Afu and Taq ligases, we find that: (a) for 3′T:C mismatches, the ratio is greater than 21 (1.51/0.07), (b) for 3′A:C mismatches, the ratio is greater than 33 (1.68/0.05), and (c) for 3′C:A mismatches, the ratio is 23 (0.92/0.04). Thus, under these reaction conditions and using these templates and probes, Afu ligase efficiently misligates 3′T:C, 3′A:C, 3′C:A, and 3′C:T mismatches compared to either of the Thermus species ligases. Conversely, each of the Thermus species ligases by definition efficiently misligate the 3′T:G mismatch compared to Afu ligase (i.e., 0.25 or 0.22 compared to no detectable misligation product).

### EXAMPLE 5

### Ligase Fidelity Analysis Using Y-Chromosome SNPs

To further evaluate the fidelity of Afu ligase relative to three Thermus species ligases (Taq ligase, AK16D ligase and T. scotoductus (Tsc) ligase (Roche)) a set of five of human Y-chromosome SNP sites were selected and corresponding ligation probe sets synthesized. The synthetic Y-chromosome template sequences are shown in Table 4 (SNP sites shown underlined).

TABLE 4

Synthetic Y-chromosome
Templates (in 3′ => 5′ orientation).

| | |
|---|---|
| Y-SNP | Template sequence |

| | | |
|---|---|---|
| YT37C1 | GCGACACTGCGCACGATTTCATGGAAA | (SEQ ID NO:10) |
| | CAACA | |
| YT37G2 | GCGACACTGCGCAGGATTTCATGGAAA | (SEQ ID NO:11) |
| | CAACA | |
| YT40A1 | AGTTTATAGGTCAAATATCTACAGCAA | (SEQ ID NO:12) |
| | ACTCTTCACCGCC | |
| YT40T2 | AGTTTATAGGTCAAATATCTACTGCAA | (SEQ ID NO:13) |
| | ACTCTTCACCGCC | |
| YT46C1 | TCAGCACAAAAGCCTAACAGAGAAAAA | (SEQ ID NO:14) |
| | CTTCAAACCTAA | |
| YT46T2 | TCAGCACAAAAGCCTAATAGAGAAAAA | (SEQ ID NO:15) |
| | CTTCAAACCTAA | |
| YT67RA1 | GCACTGTTGTAAAGCCTGAGTATTTTA | (SEQ ID NO:16) |
| | CTTGGCAGCT | |
| YT67RC2 | GCACTGTTGTAAAGCCTGCGTATTTTA | (SEQ ID NO:17) |
| | CTTGGCAGCT | |
| YT84A1 | CAGGCAAAGTGAGAGATAAGATTTTTG | (SEQ ID NO:18) |
| | TACATAACCTTAG | |
| YT84G2 | CAGGCAAAGTGAGAGATGAGATTTTTG | (SEQ ID NO:19) |
| | TACATAACCTTAG | |

Each probe set comprised two upstream probes (referred to ASO1 and ASO2 in this example), each being complementary with one of the two SNP alleles and one downstream probe (referred to as LSO in this example). As shown in Table 5, the two ASOs in a given probe set each had a different nucleotide on its 3′-end (underlined) to allow complementarity with its corresponding allele and the two probes; additionally, the probes in each probe set differed from each other based on mobility modifiers (shown in brackets). The LSO in each ligation probe set comprised a fluorescent reporter group (DR110, DTAMRA, dR6G; see U.S. Pat. No. 6,025,505).

TABLE 5
OLA Probes for Y-Chromosome SNP analysis.

| | dsSNP | | | | |
|---|---|---|---|---|---|
| SNP | dbase* | ASO1 | ASO2 | LSO | |
| Y37 | rs2032653 | TGTTGTTTCCATGA | [t]₁₂ GTTTC | pTGCGCAGTGTCGC | (SEQ ID NO:22) |
| | | AATCC | ATGAAATCG | -DR110 | |
| | | (SEQ ID NO:20; probe 20) | (SEQ ID NO:21; probe 21) | | |
| Y40 | rs2072422 | [T]₂₂ GCGGTGAAGAG | [T]₁₀ GCGGTGA | pGTAGATATTTGACCT | (SEQ ID NO:25) |
| | | TTTGCA | AGAGTTTGCT | ATAAACT- DTAMRA | |
| | | (SEQ ID NO:23; probe 23) | (SEQ ID NO:24; probe 24) | | |
| Y46 | rs21996155 | [T]₅TAGGTTTGAAGTTT | [T]₁₇ TTAGGTTTGA | pTTAGGCTTTTGTGCT | (SEQ ID NO:28) |
| | | TTCTCTA | AGTTTTTCTCTG | GA-DR110 | |
| | | (SEQ ID NO:26: probe 26) | (SEQ ID NO:27; probe 27) | | |
| Y67 | rs1558843 | AGCTGCCAAGTAAA | [T]₁₂CAAGTAAAAT | pCAGGCTTTACAACA | (SEQ ID NO:31) |
| | | ATACT | ACG | GTGC-dR6G | |
| | | (SEQ ID NO:29; probe 29) | (SEQ ID NO:30; probe 30) | | |
| Y84 | rs2032652 | CTAAGGTTATGTACAAA | [T]₁₁ CTAAGGTTAT | pATCTCTCACTTTGCC | (SEQ ID NO:34) |
| | | AATCTC | GTACAAAAATCTT | TG-DTAMRA | |
| | | (SEQ ID NO:32; probe 32) | (SEQ ID NO:33; probe 33) | | |

* the dsSNP database is found on the world wide web at ncbi.nlm.nih.gov/SNP/index

Ligation reaction compositions and reactions were performed as described in Example 4, except that the templates and corresponding probes shown in Tables 4 and 5 were used. As shown in Table 6, Afu ligase displays lower fidelity in this ligation assay under these conditions than any of the Thermus species ligases with respect to 3′C:C and 3′T:C mismatch ligations. In contrast, the Thermus species ligases generally display lower fidelity in this assay under these conditions than Afu ligase with respect to 3′G:T and 3′T:G mismatch ligations.

TABLE 6
Relative rates of mismatch ligation for the Y-Chromosome
Panel (normalized to the rate of matched ligation
using the corresponding matched ASO).

| Y-SNP | Probe No. | | Taq | AK16D | Tsc | Afu |
|---|---|---|---|---|---|---|
| (SNP nt) | (3′nt) | Mismatch | ligase | ligase | ligase | ligase |
| YT37(C) | 20 (C) | 3′C:C | — | — | — | 0.16% |
| YT37(G) | 21 (G) | 3′G:G | — | — | — | — |
| YT40(A) | 23 (A) | 3′A:A | — | — | — | — |
| YT40(T) | 24 (T) | 3′T:T | — | — | — | — |
| YT46(C) | 26 (A) | 3′A:C | — | — | 0.08% | — |
| YT46(T) | 27 (G) | 3′G:T | 4.21% | 0.32% | 0.13% | — |
| YT67(A) | 30 (G) | 3′G:A | — | — | — | — |
| YT67(C) | 29 (T) | 3′T:C | — | — | — | 0.33% |
| YT84(A) | 33 (C) | 3′C:A | 2.85% | — | — | — |
| YT84(G) | 34 (T) | 3′T:G | 1.80% | 1.26% | — | — |

### EXAMPLE 7

### Ligation Fidelity Analysis Using gDNA in a Multiplexed PCR-OLA

To evaluate the fidelity of Afu ligase relative to Taq ligase using gDNA templates, ligation probe sets corresponding to nine Y-chromosome SNPs were synthesized as described in Example 3. Four gDNA samples were obtained from the Coriell Cell Repository, Camden, N.J. (sample #35: NA 15506; sample #41: NA15283; sample #45: NA15025; and sample #158: NA 15323). The nine Y-chromosome SNPs and corresponding ligation probe sets are shown in Tables 7 A and B. Mobility modifiers, present on certain of the ASOs, are shown in brackets with subscript numbers representing the number of repeat nucleotides present in the mobility modifier. The phosphorylated LSO probes comprised the fluorescent reporter groups dR6G, dROX, dTAMRA, and dR110, as indicated in Table 7 B (see U.S. Pat. No. 6,025,505).

TABLE 7 A
Y-Chromosome SNP Probe Sets (ASO probes)

| | | | | |
|---|---|---|---|---|
| | | SNP | | |
| SNP | designation# | Alleles | ASO1 | ASO2 |

| | | | | | | |
|---|---|---|---|---|---|---|
| Y1 | rs3894 | C, T | [T]₁₇GTCACCTCTGGGACT | (SEQ ID NO:35) | [T]₁₉ GTCACCTCTGGGACT | (SEQ ID NO:36) |
| | | | GAT | GAC | | |
| Y6 | rs2032673 | C, T | TGTTTACACTCCTGAAAC | (SEQ ID NO:37) | CTGTTTACACTCCTGAAAT | (SEQ ID NO:38) |
| Y10 | M122 | C, T | [T]₄ATTTTCCCCTGAGAG | (SEQ ID NO:39) | [T]₆ ATTTTCCCCTGAGAG | (SEQ ID NO:40) |
| | | | CA | | CG | |
| Y16 | rs2032598 | C, T | [T]₅CAGCAATTTAGTATTG | (SEQ ID NO:41) | [T]₇CAGCAATTTAGTATTG | (SEQ ID NO:42) |
| | | | CCC | | CCT | |
| Y20 | rs2020857 | C, T | [T]₂₄ CAATTAATATTTTTG | (SEQ ID NO:43) | [T]₂₆ CAATTAATATTTTTG | (SEQ ID NO:44) |
| | | | AAAGGC | | AAAGAGT | |
| Y40s1a | rs2072422 | A T | [T]₁₇GCGGTGAAGAGTTTG | (SEQ ID NO:45) | [T]₂₂ gCGGTGAAGAGTTTG | (SEQ ID NO:46) |
| | | | CA | | CT | |
| Y46s1a | rs2196155 | A, G | [T]₁₇AGGTTTGAAGTTTTT | (SEQ ID NO:47) | [T]₁₉AGGTTTGAAGTTTTT | (SEQ ID NO:48) |
| | | | CTCTA | | CTCTG | |
| Y68s1 | rs3912283 | A, C | TTAATGGAGCTGAGTTCCA | (SEQ ID NO:49) | [T]₄AATGGAGCTGAGTTC (SEQ ID NO:50) | |
| | | | | | CC | |
| Y84s1a | rs2032652 | C, T | [T]₉CTAAGGTTATGTACAA | (SEQ ID NO:51) | [T]₁₁CTAAGGTTATGTACA | (SEQ ID NO:52) |
| | | | AAATCTC | AAAATCTT | | |

#:rs numbers: dsSNP database (see above); M122: see Underhill et al., Ann. Hum. Genet. 65:43-62, 2001.

TABLE 7 B

Y-Chromosome SNP Probe
Sets (labeled LSO probes)

| | |
|---|---|
| SNP | LSO probes |

| | | |
|---|---|---|
| Y1 | pAATTAGGAAGAGCTGGTACC-dR6G | (SEQ ID NO:53) |
| Y6 | pAAAATATATTTCAGCAAGACA-dR6G | (SEQ ID NO:54) |
| Y10 | pTGAATTAGTATCTCAATTGC-dROX | (SEQ ID NO:55) |
| Y16 | pGACTTTTACTAATGCATGTG-dTAMRA | (SEQ ID NO:56) |
| Y20 | pTCTTTTAGGTTAATTTAAGTACA- | (SEQ ID NO:57) |
| | dR110 | |
| Y40s1a | pGTAGATATTTGACCTATAAACT- | (SEQ ID NO:58) |
| | dTAMRA | |
| Y46s1a | pTTAGGCTTTTGTGCTGA-dR110 | (SEQ ID NO:59) |
| Y68s1 | pGTCAATATTCCCACTGATT-dR110 | (SEQ ID NO:60) |
| YB4s1a | pATCTCTCACTTTGCCTG-dTAMRA | (SEQ ID NO:61) |

The nine SNPs listed in Table 7 were interrogated using the corresponding ligation probe sets in a multiplex PCR-OLA. First, a multiplex PCR amplification was performed using the nine forward and reverse primer sets shown in Table 8A. Ten microliter amplification compositions, comprising 1 μL gDNA (1 ng/μL), 0.5 μL 25 mM MgCl₂, 4 μL AmpFISTR® PCR Reaction Mix (Applied Biosystems Part Number 361680), 1 μL 10× PCR primer mix (shown in Table 8B), 0.2 μL AmpliTaq Gold (5 U/μL), and 3.3 μL water were prepared. The amplification compositions were heated to 95° C. for eleven minutes, thermocycled for thirty-four cycles (95° C. for 30 seconds, 59° C. for 30 seconds, and 72° C. for one minute), heated to 72° C. for ten minutes, then cooled to 4° C.

TABLE 8A
Multiplex PCR Primer Sets

| | | |
|---|---|---|
| SNP | forward primer | reverse primer |

| | | | | |
|---|---|---|---|---|
| Y1 | AAATTGTGAATCTGAAATTT | (SEQ ID NO:62) | TTTCAAATAGGTTGACCTGA | (SEQ ID NO:63) |
| | AAGGG | | CAA | |
| Y6 | CTGTTCAAATCCAAAAGCT | (SEQ ID NO:64) | AAAAATTTATCTCCCCTTAG | (SEQ ID NO:65) |
| | | | CTCT | |
| Y10 | TTTTGGAAATGAATAAATCA | (SEQ ID NO:66) | CTGTGTTAGAAAAGATAGC | (SEQ ID NO:67) |
| | AGGT | | TTTATTCAG | |
| Y16 | AGAAAATTTTTGGTAACCCT | (SEQ ID NO:68) | AAAAATTCTTGGTAAGATTT | (SEQ ID NO:69) |
| | TAT | | CTCTACAT | |
| Y20 | AACTTACACTCTTTAAGCCA | (SEQ ID NO:70) | TAAACATTACATGAGAAATT | (SEQ ID NO:71) |
| | TTCC | | GCTGTAC | |
| Y40 | CACGCATCAGTTTATAGGTC | (SEQ ID NO:72) | GGTGTCCTAAGTGTAAGTAG | (SEQ ID NO:73) |
| | AAAT | | CAAGTAA | |
| Y46 | TTCTTTATCTGATTATATGT | (SEQ ID NO:74) | TTAGAACTCACAAAACTGTA | (SEQ ID NO:75) |
| | TTGCATTG | | ATCCC | |
| Y68 | CGGCAACAGATTAGAAACTA | (SEQ ID NO:76) | TTTATTCAGTGTCTGAGGTT | (SEQ ID NO:77) |
| | TG | | ACTGTAGT | |
| Y84 | TCAGCTTCCTGGATTCAGC | (SEQ ID NO:78) | GATCACCAGCAAAGGTAGCT | (SEQ ID NO:79) |

TABLE 8B
10× PCR primer mix formulations.

| | PCR primer | final OLA probe |
|---|---|---|
| Y-SNP | (pmole/10 μL) | concentration |
| Y1 | 10 | 22.2 |
| Y6 | 10 | 77.8 |
| Y10 | 16 | 888.9 |
| Y16 | 16 | 666.7 |
| Y20 | 10 | 4.4 |
| Y37s1 | 5 | 17.8 |
| Y40s1a | 5 | 53.3 |
| Y46s1a | 5 | 4.0 |
| Y68 | 5 | 4.4 |
| Y84s1a | 5 | 255.6 |

The PCR amplified targets were used in a multiplex OLA reaction as follows. Two ligation reaction compositions (one with Afu ligase, the other with Taq ligase) were formed comprising 4 μL probe mix, 1 μL Afu or Taq ligase (each at 1 U/μL), 2 μL of the amplification reaction composition described above, 1 μL ligation buffer (appropriate for each ligase), and 2 μL water. The two multiplex ligase reaction compositions were cycles 8 times (90° C. for 5 seconds, 46.5° C. for four minutes), heated to 99° C. for ten minutes, then cooled to 4° C. to generate ligation products.

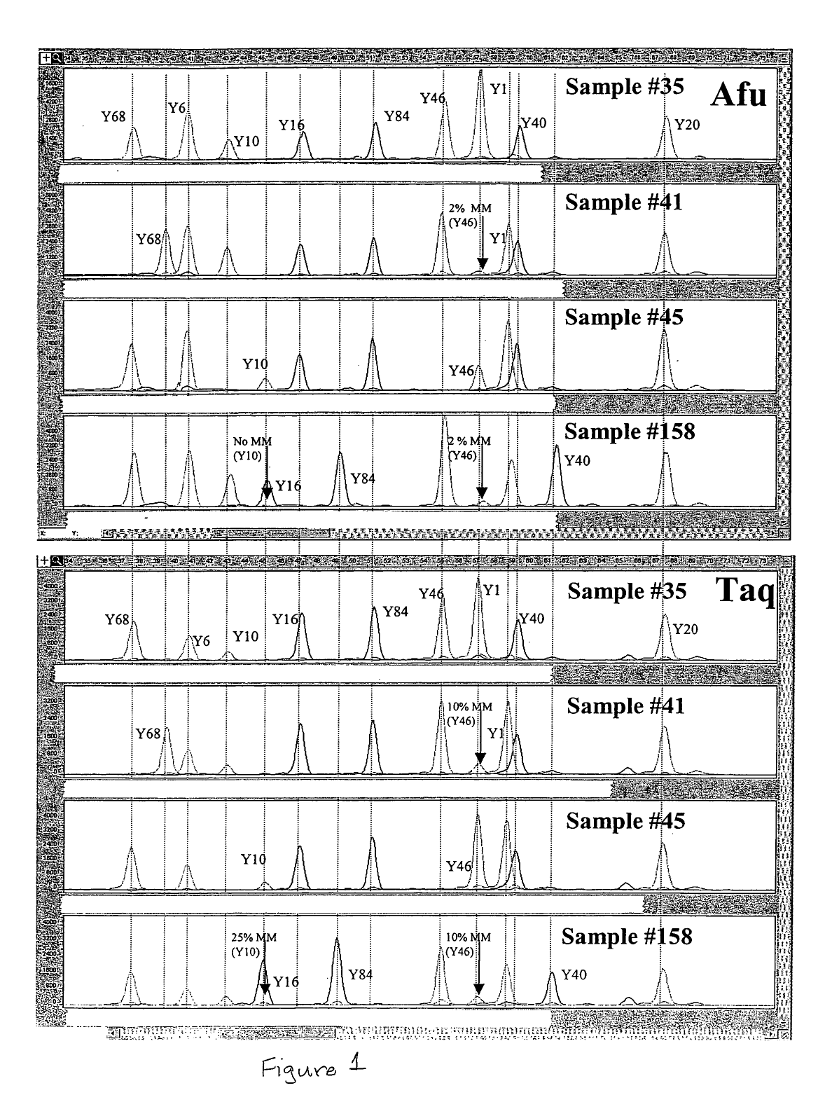

These ligation products were separated by capillary electrophoresis and analyzed as follows. Two μL of the ligation products were combined with 7.5 μL Hi-Di™ formamide and 0.5 μL GS120LIZ size standard (both from Applied Biosystems). These combinations were loaded onto 36 cm capillaries comprising POP-4 polymer on an ABI PRISM® 3100 Genetic Analyzer and separated using the SNP36_POP4 default run module. As shown in FIG. 1, some Y46 misligation products were generated with two samples when Afu ligase (2%) or Taq ligase (10%) was used, while Y10 misligation products were generated only with Taq ligase (25%).

### EXAMPLE 8

### Effect of Metal Cofactors on the Fidelity of Afu Ligase

The dependence of certain ligases on particular metal cofactors reportedly varies, depending on the ligase(s). To evaluate the effectiveness of manganese and of magnesium ions to serve as cofactors for Afu ligase, a ligation assay was performed in the presence of varying concentrations of the manganese or the magnesium ions, as follows.

A DNA premix was prepared and hybridization complexes formed as described in Example 4, except that the premix comprised 400 nM 5′-phosphate-GCCAAGCTTGCATGCCTG C-3′Fam (SEQ ID NO:80), 800 nM (−21)M13-primer 5′-TGTAAAACGACGGCCAGT-3′ (SEQ ID NO:81) and 800 nM of the artificial M13-derived target 5′-ACATTTTGCTGC CGGTCACGGTTCGAACGTACGGACG-3′ (SEQ ID NO:82) in 20 mM NaCl.

For each of the two metal ions, a series of ten dilutions of either MnCl₂ or MgCl₂ was prepared (0 to 60 mM). For each metal ion series, 25 μL comprising 0.02 U/μl Afu ligase in 100 mM Tris-HCl pH 7.5, 20 mM DTT, 2 mM ATP, 0.2% Triton X-100 (2× Afu ligation buffer without metal cofactor) was placed into ten themocycler tubes in parallel (twenty tubes total). To each tube was added 20 μl of one of the metal cofactor dilutions, bringing the volume in each tube to 45 μl. Both sets of tubes were placed on a thermocycler block and maintained at 45° C.

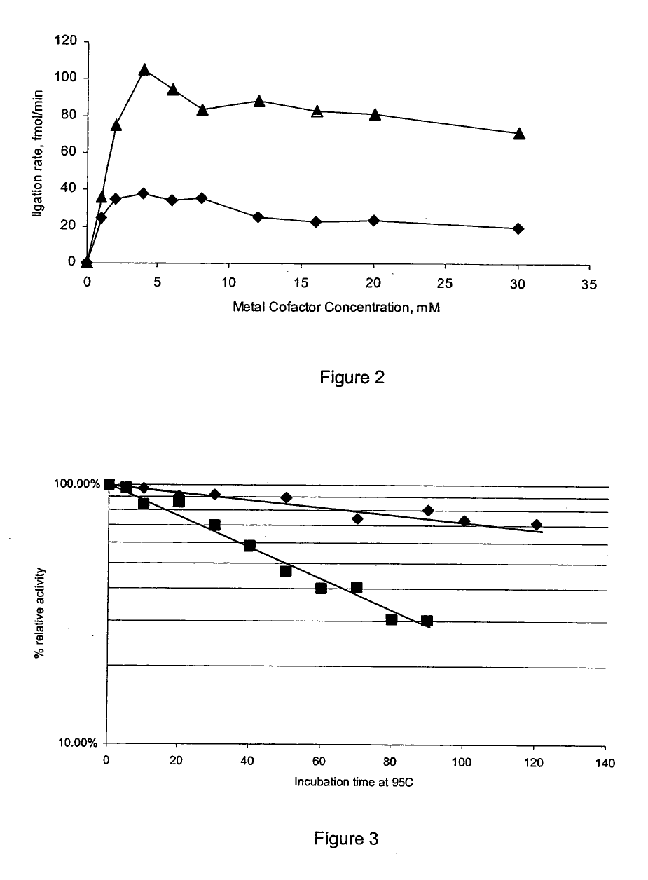

The ligation reaction was started by adding 5 μl of the DNA premix to each of the twenty parallel tubes. The resulting 50 μl ligation reaction compositions comprised 1× ligase buffer and 40 nM substrate. A 5 μL aliquot of the reaction composition comprising ligation products was combined with 10 μL of 100 mM EDTA at reaction times of 2 min., 4 min., 8 min., and 20 min. to stop the reaction and the samples were chilled on ice and-analyzed as described in Example 4. The results, shown in FIG. 2, suggest that at least under these reaction conditions, Afu ligase can utilize either Mn²⁺ or Mg²⁺ as the metal ion cofactor.

### EXAMPLE 9

### Thermal Stability Analysis of Afu Ligase

To evaluate the thermostability of Afu ligase, a side-by-side comparison with Taq ligase was performed at 95° C. and a thermal decay curve generated. A DNA premix was prepared as described in Example 8. Into two parallel sets of twelve iced tubes was placed either 25 μl of 0.02 U/μl Afu- or Taq ligase in the appropriate ligation buffer (one set of twelve tubes for each ligase). One tube from each set was retained on ice (t₀ sample) and the remaining eleven tubes were placed in a thermocycler at temperature of 95° C. One tube from each set was periodically removed from the thermocycler over a two-hour time course and placed on ice. At the end of the two-hour time course, 20 μl of buffer containing 2.5 μl of the appropriate 10× ligation buffer were added to each tube bringing the volume to 45 μl. The tubes were placed on a thermocycler block and maintained at 45° C. Five μL of DNA premix was added to each tube to start generating ligation products. A 5 μL aliquot of the reaction composition comprising ligation products was combined with 10 μL of 100 mM EDTA at reaction times of 2 min., 8 min., 20 min., and 60 min. to stop the reaction and the samples were chilled on ice separated and analyzed as described in Example 4. The thermal decay curve is shown in FIG. 3.

Although the disclosed teachings has been described with reference to various applications, methods, and compositions, it will be appreciated that various changes and modifications may be made without departing from the teachings herein. The foregoing examples are provided to better illustrate the disclosed teachings and are not intended to limit the scope of the teachings herein.

1. An isolated thermostable polypeptide that efficiently misligates adjacently hybridized suitable oligonucleotides when the nucleotide on the 3′ terminus of the upstream probe is a T and the corresponding template nucleotide is a C, but does not efficiently misligate adjacently hybridized suitable oligonucleotides when the nucleotide on the 3′ terminus of the upstream probe is a T and the corresponding template nucleotide is a G.

2. The isolated polypeptide of claim 1, wherein the efficiently misligates refers to a ligation rate ratio of at least 5:1 relative to that of Taq ligase or Thermus species AK16D ligase.

3. The isolated polypeptide of claim 2, wherein the efficiently misligates refers to a ligation rate ratio is at least 10:1.

4. The isolated polypeptide of claim 2, wherein the efficiently misligates refers to a ligation rate ratio is at least 20:1.

5. The isolated polypeptide of claim 1, wherein the does not efficiently misligate refers to a ligation rate ratio is less than 1:10 relative to that of Taq ligase or Thermus species AK16D ligase.

6. The isolated polypeptide of claim 5, wherein the does not efficiently misligate refers to a ligation rate ratio is less than 1:20.

7. The isolated polypeptide of claim 5, wherein the does not efficiently misligate refers to a ligation rate ratio is less than 1:30.

8. The isolated polypeptide of claim 5, wherein the efficiently misligates refers to a ligation rate ratio of at least 5:1 relative to that of Taq ligase or Thermus species AK16D ligase.

9. The isolated polypeptide of claim 5, wherein the efficiently misligates refers to a ligation rate ratio of at least 10:1 relative to that of Taq ligase or Thermus species AK16D ligase.

10. The isolated polypeptide of claim 5, wherein the efficiently misligates refers to a ligation rate ratio of at least 20:1 relative to that of Taq ligase or Thermus species AK16D ligase.

11. The isolated polypeptide of claim 6, wherein the efficiently misligates refers to a ligation rate ratio of at least 10:1 relative to that of Taq ligase or Thermus species AK16D ligase.

12. The isolated polypeptide of claim 6, wherein the efficiently misligates refers to a ligation rate ratio of at least 20:1 relative to that of Taq ligase or Thermus species AK16D ligase.

13. The isolated polypeptide of claim 12, wherein the polypeptide is derived from Archaeoglobus fulgidus.

14. The isolated polypeptide of claim 7, wherein the efficiently misligates refers to a ligation rate ratio of at least 10:1 relative to that of Taq ligase or Thermus species AK16D ligase.

15. The isolated polypeptide of claim 14, wherein the polypeptide is derived from Archaeoglobus fulgidus.

16. The isolated polypeptide of claim 7, wherein the efficiently misligates refers to a ligation rate ratio of at least 20:1 relative to that of Taq ligase or Thermus species AK16D ligase.

17. The isolated polypeptide of claim 16, wherein the polypeptide is derived from Archaeoglobus fulgidus.

18. A method for generating a ligation product comprising:
forming a ligation reaction composition comprising at least one target nucleic acid sequence comprising; at least one ligation probe set comprising at least one first probe and at least one second probe, wherein the at least one first probe comprises at least one first target-specific portion and the at least one second probe comprises at least one second target-specific portion; and Afu ligase, including enzymatically active fragment or variants thereof; and
subjecting the ligation reaction composition to at least one cycle of ligation to generate at least one ligation product.

19. The method of claim 18, further comprising amplifying the at least one target nucleic acid sequence, the at least one ligation product, or the at least one target nucleic acid sequence and the at least one ligation product.

20. The method of claim 18, wherein the at least one first probe and the at least one second probe hybridize adjacently on the at least one target nucleic acid sequence.

21. The method of claim 18, wherein the at least one first probe and the at least one second probe do not hybridize adjacently on the at least one target nucleic acid sequence, and further comprising extending at least one hybridized probe.

22. A method for generating a misligation product comprising:
forming a ligation reaction composition comprising at least one target nucleic acid sequence; at least one ligation probe set comprising at least one first probe and at least one second probe, wherein the at least one first probe comprises at least one first target-specific portion and the at least one second probe comprises at least one second target-specific portion and wherein the target-specific portion of at least one probe comprises at least one nucleotide mismatch relative to the target nucleic acid sequence; and Afu ligase, including enzymatically active fragment or variants thereof; and
subjecting the ligation reaction composition to at least one cycle of ligation to generate at least one misligation product.

23. The method of claim 22, further comprising amplifying the at least one target nucleic acid sequence, the at least one ligation product, or the at least one target nucleic acid sequence and the at least one ligation product.

24. The method of claim 22, wherein the at least one first probe and the at least one second probe hybridize adjacently on the at least one target nucleic acid sequence.

25. The method of claim 22, wherein the at least one first probe and the at least one second probe do not hybridize adjacently on the at least one target nucleic acid sequence, and further comprising extending at least one hybridized probe.

26. A method for generating at least one ligation product, comprising:
at least one step for interrogating at least one target nucleotide; and
at least one step for generating at least one ligation product.

27. The method of claim 26, further comprising at least one step for amplifying the at least one ligation product.

28. The method of claim 27, further comprising at least one step for digesting the at least one amplified ligation product.

29. The method of claim 26, further comprising at least one step for digesting the at least one ligation product.

30. A method for generating at least one misligation product, comprising:
at least one step for interrogating at least one target nucleotide; and
at least one step for generating at least one ligation product.

31. The method of claim 30, further comprising at least one step for amplifying the at least one ligation product.

32. The method of claim 31, further comprising at least one step for digesting the at least one amplified ligation product.

33. The method of claim 30, further comprising at least one step for digesting the at least one ligation product.

34. A method for identifying at least two target nucleotides in a sample, comprising:
forming a first ligation reaction composition comprising (a) at least one first target nucleic acid sequence, (b) at least one first ligation probe set comprising at least one first probe and at least one second probe, wherein the at least one first probe comprises at least one first target-specific portion and the at least one second probe comprises at least one second target-specific portion, and (c) at least one first ligase;
forming a second ligation reaction composition comprising (a) at least one second target nucleic acid sequence, (b) at least one second ligation probe set comprising at least one first probe and at least one second probe, wherein the at least one first probe comprises at least one target-specific portion and the at least one second probe comprises at least one target-specific portion, and (c) at least one second ligase;
subjecting the first ligation reaction composition to at least one cycle of ligation to generate at least one first ligation product;
subjecting the second ligation reaction composition to at least one cycle of ligation to generate at least one second ligation product; and
identifying the at least one first target nucleotide, the at least one second target nucleotide, or the at least one first target nucleotide and the at least one second target nucleotide in the sample.

35. The method of claim 34, wherein the subjecting the first ligation reaction composition to at least one cycle of ligation and the subjecting the second ligation reaction composition to at least one cycle of ligation are performed in parallel.

36. The method of claim 34, wherein the subjecting the first ligation reaction composition to at least one cycle of ligation and the subjecting the second ligation reaction composition to at least one cycle of ligation are performed separately.

37. The method of claim 34, wherein the forming the first ligation reaction composition and the forming the second ligation reaction composition are performed in parallel.

38. The method of claim 34, wherein the forming the first ligation reaction composition and the forming the second ligation reaction composition are performed separately.

39. The method of claim 34, wherein the at least one first ligation product, the at least one second ligation product, or the at least one first ligation product and the at least one second ligation product, further comprises at least one primer-binding portion, at least one reporter group, at least one mobility modifier, at least one hybridization tag, at least one reporter probe binding portion, at least one affinity tag, or combinations thereof.

40. The method of claim 39, wherein the identifying comprises detecting the at least one reporter group of the at least one first ligation product and the at least one reporter group of the at least one second ligation product, and comparing the ratio of the at least one first ligation product with the ratio of at least one second ligation product.

41. The method of claim 34, wherein the at least one cycle of ligation comprises a multiplicity of cycles of ligation.

42. The method of claim 34, further comprising amplifying the at least one first ligation product, the at least one second ligation product, or the at least one first ligation product and the at least one second ligation product to generate at least one amplified first ligation product, at least one amplified second ligation product, or at least one amplified first ligation product and at least one amplified second ligation product.

43. The method of claim 42, wherein the at least one amplified first ligation product, the at least one amplified second ligation product, or the at least one amplified first ligation product and the at least one amplified second ligation product, further comprises at least one primer-binding portion, at least one reporter group, at least one mobility modifier, at least one hybridization tag, at least one reporter probe-binding portion, at least one affinity tag, or combinations thereof.

44. The method of claim 43, wherein the identifying comprises detecting the at least one reporter group of the at least one amplified first ligation product and the at least one reporter group of the at least one amplified second ligation product, and comparing the ratio of the at least one amplified first ligation product with the ratio of at least one amplified second ligation product.

45. The method of claim 34, wherein the at least one first ligase comprises Afu ligase, including enzymatically active mutants or variants thereof.

46. The method of claim 45, wherein the at least one second ligase comprises at least one Thermus species ligase.

47. A kit comprising the polypeptide of claim 1.

48. The kit of claim 47, further comprising at least one probe set, at least one primer set, at least one reporter group, at least one affinity tag, at least one hybridization tag, at least one mobility modifier, at least one polymerase, at least one nuclease, or combinations thereof.

49. A kit comprising the polypeptide of claim 15.

50. The kit of claim 49, further comprising at least one probe set, at least one primer set, at least one reporter group, at least one affinity tag, at least one hybridization tag, at least one mobility modifier, at least one polymerase, at least one nuclease, or combinations thereof.

51. A kit comprising the polypeptide of claim 17.

52. The kit of claim 51, further comprising at least one probe set, at least one primer set, at least one reporter group, at least one affinity tag, at least one hybridization tag, at least one mobility modifier, at least one polymerase, at least one nuclease, or combinations thereof.

53. The polypeptide of claim 1, wherein the polypeptide is expressed recombinantly.

54. The polypeptide of claim 15, wherein the polypeptide is expressed recombinantly.

55. The polypeptide of claim 17, wherein the polypeptide is expressed recombinantly.

56. The method of claim 26, further comprising Afu ligase.

57. The method of claim 30, further comprising Afu ligase.


SEQUENCE LISTING (84 sequences)

SEQ ID NO: 1 - DNA, 37 bp, Artificial Sequence
  Description of Artificial Sequence Synthetic oligonucleotide
acattttgctgccggtcacggttcgaacgtacggacg

SEQ ID NO: 2 - DNA, 37 bp, Artificial Sequence
  Description of Artificial Sequence Synthetic oligonucleotide
acattttgctgccggtctcggttcgaacgtacggacg

SEQ ID NO: 3 - DNA, 37 bp, Artificial Sequence
  Description of Artificial Sequence Synthetic oligonucleotide
acattttgctgccggtcgcggttcgaacgtacggacg

SEQ ID NO: 4 - DNA, 37 bp, Artificial Sequence
  Description of Artificial Sequence Synthetic oligonucleotide
acattttgctgccggtcccggttcgaacgtacggacg

SEQ ID NO: 5 - DNA, 18 bp, Artificial Sequence
  Description of Artificial Sequence Synthetic probe
tgtaaaacgacggccagt

SEQ ID NO: 6 - DNA, 15 bp, Artificial Sequence
  Description of Artificial Sequence Synthetic probe
gccaagcttcgatgc

SEQ ID NO: 7 - DNA, 22 bp, Artificial Sequence
  Description of Artificial Sequence Synthetic probe
tttttgtaaaacgacggccaga

SEQ ID NO: 8 - DNA, 26 bp, Artificial Sequence
  Description of Artificial Sequence Synthetic probe
tttttttttgtaaaacgacggccagc

SEQ ID NO: 9 - DNA, 30 bp, Artificial Sequence
  Description of Artificial Sequence Synthetic probe
tttttttttttttgtaaaacgacggccagg

SEQ ID NO: 10 - DNA, 32 bp, Artificial Sequence
  Description of Artificial Sequence Synthetic oligonucleotide
acaacaaaggtactttagcacgcgtcacagcg

SEQ ID NO: 11 - DNA, 32 bp, Artificial Sequence
  Description of Artificial Sequence Synthetic oligonucleotide
acaacaaaggtactttaggacgcgtcacagcg

SEQ ID NO: 12 - DNA, 40 bp, Artificial Sequence
  Description of Artificial Sequence Synthetic oligonucleotide
ccgccacttctcaaacgacatctataaactggatatttga

SEQ ID NO: 13 - DNA, 40 bp, Artificial Sequence
  Description of Artificial Sequence Synthetic oligonucleotide
ccgccacttctcaaacgtcatctataaactggatatttga

SEQ ID NO: 14 - DNA, 39 bp, Artificial Sequence
  Description of Artificial Sequence Synthetic oligonucleotide
aatccaaacttcaaaaagagacaatccgaaaacacgact

SEQ ID NO: 15 - DNA, 39 bp, Artificial Sequence
  Description of Artificial Sequence Synthetic oligonucleotide
aatccaaacttcaaaaagagataatccgaaaacacgact

SEQ ID NO: 16 - DNA, 37 bp, Artificial Sequence
  Description of Artificial Sequence Synthetic oligonucleotide
tcgacggttcattttatgagtccgaaatgttgtcacg

SEQ ID NO: 17 - DNA, 37 bp, Artificial Sequence
  Description of Artificial Sequence Synthetic oligonucleotide
tcgacggttcattttatgcgtccgaaatgttgtcacg

SEQ ID NO: 18 - DNA, 40 bp, Artificial Sequence
  Description of Artificial Sequence Synthetic oligonucleotide
gattccaatacatgtttttagaatagagagtgaaacggac

SEQ ID NO: 19 - DNA, 40 bp, Artificial Sequence
  Description of Artificial Sequence Synthetic oligonucleotide
gattccaatacatgtttttagagtagagagtgaaacggac

SEQ ID NO: 20 - DNA, 19 bp, Artificial Sequence
  Description of Artificial Sequence Synthetic probe
tgttgtttccatgaaatcc

SEQ ID NO: 21 - DNA, 26 bp, Artificial Sequence
  Description of Artificial Sequence Synthetic probe
ttttttttttttgtttcatgaaatcg

SEQ ID NO: 22 - DNA, 13 bp, Artificial Sequence
  Description of Artificial Sequence Synthetic probe
tgcgcagtgtcgc

SEQ ID NO: 23 - DNA, 39 bp, Artificial Sequence
  Description of Artificial Sequence Synthetic probe
ttttttttttttttttttttttgcggtgaagagtttgca

SEQ ID NO: 24 - DNA, 27 bp, Artificial Sequence
  Description of Artificial Sequence Synthetic probe
ttttttttttgcggtgaagagtttgct

SEQ ID NO: 25 - DNA, 22 bp, Artificial Sequence
  Description of Artificial Sequence Synthetic probe
gtagatatttgacctataaact

SEQ ID NO: 26 - DNA, 27 bp, Artificial Sequence
  Description of Artificial Sequence Synthetic probe
tttttttaggtttgaagtttttctcta

SEQ ID NO: 27 - DNA, 39 bp, Artificial Sequence
  Description of Artificial Sequence Synthetic probe
tttttttttttttttttttaggtttgaagtttttctctg

SEQ ID NO: 28 - DNA, 17 bp, Artificial Sequence
  Description of Artificial Sequence Synthetic probe
ttaggcttttgtgctga

SEQ ID NO: 29 - DNA, 19 bp, Artificial Sequence
  Description of Artificial Sequence Synthetic probe
agctgccaagtaaaatact

SEQ ID NO: 30 - DNA, 25 bp, Artificial Sequence
  Description of Artificial Sequence Synthetic probe
ttttttttttttcaagtaaaatacg

SEQ ID NO: 31 - DNA, 18 bp, Artificial Sequence
  Description of Artificial Sequence Synthetic probe
caggctttacaacagtgc

SEQ ID NO: 32 - DNA, 23 bp, Artificial Sequence
  Description of Artificial Sequence Synthetic probe
ctaaggttatgtacaaaaatctc

SEQ ID NO: 33 - DNA, 34 bp, Artificial Sequence
  Description of Artificial Sequence Synthetic probe
tttttttttttctaaggttatgtacaaaaatctt

SEQ ID NO: 34 - DNA, 17 bp, Artificial Sequence
  Description of Artificial Sequence Synthetic probe
atctctcactttgcctg

SEQ ID NO: 35 - DNA, 35 bp, Artificial Sequence
  Description of Artificial Sequence Synthetic probe
tttttttttttttttttgtcacctctgggactgat

SEQ ID NO: 36 - DNA, 37 bp, Artificial Sequence
  Description of Artificial Sequence Synthetic probe
tttttttttttttttttttgtcacctctgggactgac

SEQ ID NO: 37 - DNA, 18 bp, Artificial Sequence
  Description of Artificial Sequence Synthetic probe
tgtttacactcctgaaac

SEQ ID NO: 38 - DNA, 19 bp, Artificial Sequence
  Description of Artificial Sequence Synthetic probe
ctgtttacactcctgaaat

SEQ ID NO: 39 - DNA, 21 bp, Artificial Sequence
  Description of Artificial Sequence Synthetic probe
ttttattttcccctgagagca

SEQ ID NO: 40 - DNA, 23 bp, Artificial Sequence
  Description of Artificial Sequence Synthetic probe
ttttttattttcccctgagagcg

SEQ ID NO: 41 - DNA, 24 bp, Artificial Sequence
  Description of Artificial Sequence Synthetic probe
tttttcagcaatttagtattgccc

SEQ ID NO: 42 - DNA, 26 bp, Artificial Sequence
  Description of Artificial Sequence Synthetic probe
tttttttcagcaatttagtattgcct

SEQ ID NO: 43 - DNA, 46 bp, Artificial Sequence
  Description of Artificial Sequence Synthetic probe
ttttttttttttttttttttttttcaattaatatttttgaaagagc

SEQ ID NO: 44 - DNA, 48 bp, Artificial Sequence
  Description of Artificial Sequence Synthetic probe
ttttttttttttttttttttttttttcaattaatatttttgaaagagt

SEQ ID NO: 45 - DNA, 37 bp, Artificial Sequence
  Description of Artificial Sequence Synthetic probe
ttttttttttttttttttttgcggtgaagagtttgca

SEQ ID NO: 46 - DNA, 39 bp, Artificial Sequence
  Description of Artificial Sequence Synthetic probe
ttttttttttttttttttttttgcggtgaagagtttgct

SEQ ID NO: 47 - DNA, 37 bp, Artificial Sequence
  Description of Artificial Sequence Synthetic probe
tttttttttttttttttaggtttgaagtttttctcta

SEQ ID NO: 48 - DNA, 39 bp, Artificial Sequence; identical to SEQ ID NO: 27
  Description of Artificial Sequence Synthetic probe

SEQ ID NO: 49 - DNA, 19 bp, Artificial Sequence
  Description of Artificial Sequence Synthetic probe
ttaatggagctgagttcca

SEQ ID NO: 50 - DNA, 21 bp, Artificial Sequence
  Description of Artificial Sequence Synthetic probe
ttttaatggagctgagttccc

SEQ ID NO: 51 - DNA, 32 bp, Artificial Sequence
  Description of Artificial Sequence Synthetic probe
tttttttttctaaggttatgtacaaaaatctc

SEQ ID NO: 52 - DNA, 34 bp, Artificial Sequence; identical to SEQ ID NO: 33
  Description of Artificial Sequence Synthetic probe

SEQ ID NO: 53 - DNA, 20 bp, Artificial Sequence
  Description of Artificial Sequence Synthetic probe
aattaggaagagctggtacc

SEQ ID NO: 54 - DNA, 21 bp, Artificial Sequence
  Description of Artificial Sequence Synthetic probe
aaaatatatttcagcaagaca

SEQ ID NO: 55 - DNA, 20 bp, Artificial Sequence
  Description of Artificial Sequence Synthetic probe
tgaattagtatctcaattgc

SEQ ID NO: 56 - DNA, 20 bp, Artificial Sequence
  Description of Artificial Sequence Synthetic probe
gacttttactaatgcatgtg

SEQ ID NO: 57 - DNA, 23 bp, Artificial Sequence
  Description of Artificial Sequence Synthetic probe
tcttttaggttaatttaagtaca

SEQ ID NO: 58 - DNA, 22 bp, Artificial Sequence
  Description of Artificial Sequence Synthetic probe
gtagatatttgacctataaact

SEQ ID NO: 59 - DNA, 17 bp, Artificial Sequence
  Description of Artificial Sequence Synthetic probe
ttaggcttttgtgctga

SEQ ID NO: 60 - DNA, 19 bp, Artificial Sequence
  Description of Artificial Sequence Synthetic probe
gtcaatattcccactgatt

SEQ ID NO: 61 - DNA, 17 bp, Artificial Sequence
  Description of Artificial Sequence Synthetic probe
atctctcactttgcctg

SEQ ID NO: 62 - DNA, 25 bp, Artificial Sequence
  Description of Artificial Sequence Synthetic primer
aaattgtgaatctgaaatttaaggg

SEQ ID NO: 63 - DNA, 23 bp, Artificial Sequence
  Description of Artificial Sequence Synthetic primer
tttcaaataggttgacctgacaa

SEQ ID NO: 64 - DNA, 19 bp, Artificial Sequence
  Description of Artificial Sequence Synthetic primer
ctgttcaaatccaaaagct

SEQ ID NO: 65 - DNA, 24 bp, Artificial Sequence
  Description of Artificial Sequence Synthetic primer
aaaaatttatctccccttagctct

SEQ ID NO: 66 - DNA, 24 bp, Artificial Sequence
  Description of Artificial Sequence Synthetic primer
ttttggaaatgaataaatcaaggt

SEQ ID NO: 67 - DNA, 28 bp, Artificial Sequence
  Description of Artificial Sequence Synthetic primer
ctgtgttagaaaagatagctttattcag

SEQ ID NO: 68 - DNA, 23 bp, Artificial Sequence
  Description of Artificial Sequence Synthetic primer
agaaaatttttggtaacccttat

SEQ ID NO: 69 - DNA, 28 bp, Artificial Sequence
  Description of Artificial Sequence Synthetic primer
aaaaattcttggtaagatttctctacat

SEQ ID NO: 70 - DNA, 24 bp, Artificial Sequence
  Description of Artificial Sequence Synthetic primer
aacttacactctttaagccattcc

SEQ ID NO: 71 - DNA, 27 bp, Artificial Sequence
  Description of Artificial Sequence Synthetic primer
taaacattacatgagaaattgctgtac

SEQ ID NO: 72 - DNA, 24 bp, Artificial Sequence
  Description of Artificial Sequence Synthetic primer
cacgcatcagtttataggtcaaat

SEQ ID NO: 73 - DNA, 27 bp, Artificial Sequence
  Description of Artificial Sequence Synthetic primer
ggtgtcctaagtgtaagtagcaagtaa

SEQ ID NO: 74 - DNA, 28 bp, Artificial Sequence
  Description of Artificial Sequence Synthetic primer
ttctttatctgattatatgtttgcattg

SEQ ID NO: 75 - DNA, 25 bp, Artificial Sequence
  Description of Artificial Sequence Synthetic primer
ttagaactcacaaaactgtaatccc

SEQ ID NO: 76 - DNA, 22 bp, Artificial Sequence
  Description of Artificial Sequence Synthetic primer
cggcaacagattagaaactatg

SEQ ID NO: 77 - DNA, 28 bp, Artificial Sequence
  Description of Artificial Sequence Synthetic primer
tttattcagtgtctgaggttactgtagt

SEQ ID NO: 78 - DNA, 19 bp, Artificial Sequence
  Description of Artificial Sequence Synthetic primer
tcagcttcctggattcagc

SEQ ID NO: 79 - DNA, 20 bp, Artificial Sequence
  Description of Artificial Sequence Synthetic primer
gatcaccagcaaaggtagct

SEQ ID NO: 80 - DNA, 19 bp, Artificial Sequence
  Description of Artificial Sequence Synthetic oligonucleotide
gccaagcttgcatgcctgc

SEQ ID NO: 81 - DNA, 18 bp, Artificial Sequence
  Description of Artificial Sequence Synthetic oligonucleotide
tgtaaaacgacggccagt

SEQ ID NO: 82 - DNA, 37 bp, Artificial Sequence; identical to SEQ ID NO: 1
  Description of Artificial Sequence Synthetic oligonucleotide

SEQ ID NO: 83 - DNA, 25 bp, Artificial Sequence
  Description of Artificial Sequence Synthetic primer
catatgatgttgtttgccgagtttg

SEQ ID NO: 84 - DNA, 29 bp, Artificial Sequence
  Description of Artificial Sequence Synthetic primer
tcgactcattgtctctttacctcgaactg